(19) United States
(12) Patent Application Publication
(10) Pub. No.: US 2024/0335827 A1
(43) Pub. Date: Oct. 10, 2024

(54) IRON-BASED COMPLEXES FOR USE IN THE CATALYSIS OF HYDROSILYLATION REACTIONS

(71) Applicant: THE GOVERNING COUNCIL OF THE UNIVERSITY OF TORONTO, Toronto, Ontario (CA)

(72) Inventors: Datong Song, Toronto, Ontario (CA); Qiuming Liang, Toronto, Ontario (CA); José Luis Jimenez Santiago, Toronto, Ontario (CA)

(21) Appl. No.: 18/573,316

(22) PCT Filed: Jun. 23, 2022
(86) PCT No.: PCT/CA2022/051014

§ 371 Date: Dec. 21, 2023

Related U.S. Application Data
(60) Provisional application No. 63/202,810, filed on Jun. 25, 2021.

(51) Int. Cl. B01J 31/22; B01J 37/00; C07F 7/08; C07F 15/02

(52) U.S. Cl. CPC B01J 31/2273; B01J 37/00; C07F 7/0876; C07F 15/02; B01J 2231/323; B01J 2531/842

(57) ABSTRACT

There is provided an iron-based complex of formula (IA) or (IB) or a solvate thereof (IA) (IB). The iron-based complex can be used for catalyzing a hydrosillation reaction, such as the hydrosilylation of a ketone or an aldehyde. Also provided is a method for preparing a silyl ether from a ketone or an aldehyde comprising the hydrosilylation of the ketone or aldehyde in the presence of the iron-based complex. Another method is provided for the synthesis of an alcohol from a ketone or an aldehyde comprising the hydrosilylation of the ketone or aldehyde in the presence of the iron-based complex to form a silyl ether, and then hydrolyzing the silyl ether to obtain the alcohol. A process for preparing the iron-based complex is further disclosed.

### TECHNICAL FIELD

The technical field generally relates to iron-based complexes useful in the catalysis of hydrosilylation reactions. More particularly, the technical field relates to new iron-based complexes and their process of synthesis, their use as catalysts in the synthesis of alcohols from ketone and aldehydes involving a hydrosilylation reaction.

### BACKGROUND

The reduction of ketone and aldehyde functional groups into the corresponding alcohol or alcohol derivatives is a highly important chemical transformation in the agrochemical and pharmaceutical industry (Riener K., Hoegerl M. P., Gigler P., Kuehn F. E. ACS Catal. 2012, 2, 613-621; Raya-Baron A., Ona-Burgos P., Fernandez I. ACS Catal. 2019, 9, 5400-5417). Two common ways to reduce ketones and aldehydes into alcohols are the stoichiometric reduction using highly reactive aluminum or boron hydride reagents and catalytic hydrogenation. The former involves highly reactive and hazardous main group hydride reagents, which is difficult to scale up safely and has poor functional group tolerance The selective catalytic hydrogenation of ketones and aldehydes features decent functional group tolerance and excellent conversions, but usually requires pressurized dihydrogen gas, giving rise to severe fire and explosion hazards as well (Wang D., Astruc D. Chem. Rev. 2015, 115, 6621-6686; Morris H. R. Chem. Soc. Rev. 2009, 38, 2282-2291). As an alternative to these methods, the catalytic hydrosilylation of ketones and aldehydes combines the safe and mild reaction conditions with a suitable reducing power. Moreover, the immediate products of the hydrosilylation of ketones and aldehydes are silyl-protected alcohols (Scheme 1), which are compatible with highly basic reagents (such as Grignard reagents), rendering the method a unique advantage over hydrogenation in a multistep synthesis.

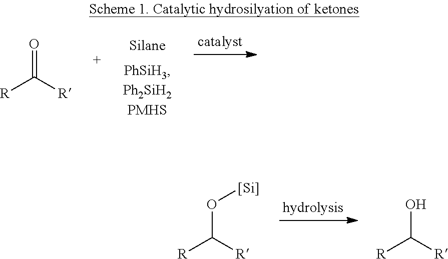

Typically, this reaction is catalyzed by scarce and costly noble metals such as Ru, Rh, Ir, and Pt and expensive reducing agents such as PhSiH₃, Ph₂SiH₂, and (EtO)₃SiH (Marciniec B., Gulinski J., Urbaniak W., Kornetka Z. W. Pergamon Press: Oxford, U.K., 1992; Roy, A. K. Adv. Organomet. Chem. 2007, 55, 1-59). Therefore, to make this protocol more attractive for the industry, it is necessary to look for more sustainable and environmentally benign systems. In recent years, replacing the costly reducing agents with the cheap and air stable PMHS (polymethylhydrosiloxane) as hydrosilane has been seen as a more attractive protocol (Lawrence N. J., Drew M. D., Bushell S. M. J. Chem. Soc., Perkin Trans. 1999, 1, 3381-3391; Pesti J., Larson G. L. Org. Process Res. Dev. 2016, 20, 1164-1181).

However, there are very few publications where PMHS is used as the reducing agent. It is worth mentioning that the best catalysts for this purpose are copper and iron-based catalysts. In 2014, Roy et al. reported a neutral copper (I) catalyst [(aNHC)Cu(Cl])] that was able to reduce a ketone at 0.25 mol % catalyst loading using PMHS (Roy S. R., Sau S. C., Mandal, S. K. J., Org. Chem. 2014, 79, 9150-9160). Later, Cazin et al. showed that the dinuclear copper-NHC complex [Cu(μ-trz) (NHC)]₂ was the most efficient catalyst using PMHS, and, upon heating the reaction at 55° C. a wide variety of ketones were converted into the alcohol within hours (3-16 h) at 0.05 mol % catalyst loading (Cazin, C. S., J. ACS Catal. 2017, 7, 238-242).

Iron besides its intrinsic characteristic of being environmentally benign and abundant, has been shown to form some highly active catalysts for this transformation. However, when PMHS is used as the reducing agent, iron-based systems require a higher catalyst loading (>0.5 mol %) and usually a higher temperature (>50° C.) (Raya-Baron A., Ona-Burgos P., Fernandez, I., ACS Catal. 2019, 9, 5400-5417). In 2013, Lopes R. et al. reported the NHC-iron complex [(Cp-NHC)Fe(CO)(OH)] was active at catalyst loading of 0.5 mol %, and complete conversion of ketones into the silyl ether were achieved at room temperature within 1-48 h (Lopes R., Cardoso J. M. S., Postigo, L., Royo, B., Catal. Lett. 2013, 143, 1061-1066).

There is still a need for new efficient catalysts for the hydrosilylation of ketones and aldehydes and the preparation of a variety of alcohols from the corresponding silyl ethers, at the industrial scale.

### SUMMARY

According to one aspect, there is provided and iron-based complex of formula (IA) or (IB) or a solvate thereof

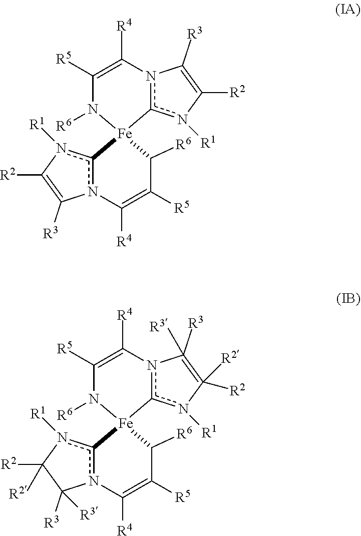

wherein:
the chain

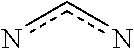

comprise one single bond and one double bond;
R¹ represents a linear or branched C₁-C₁₈alkyl, linear or branched C₂-C₁₈alkenyl, C₃C₁₀cycloalkyl, C₃C₁₀cycloalkenyl or C₆-C₁₄aryl, where the alkyl, alkenyl, cycloalkyl, cycloalkenyl and aryl are optionally substituted with one or more substituents selected from the group consisting of halogen, linear or branched C₁-C₆alkyl, linear or branched C₂-C₆alkenyl, C₃C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷, tetrahydrofuran-2-yl, tetrahydrofuran-3-yl, NO², CN, NH², OCF³, and CF³;
R², R²′, R³ and R³′, which are identical or different, represent H, a linear or branched C₁-C₁₀alkyl, linear or branched C₂-C₁₀alkenyl, C₃-C₁₀cycloalkyl, C₃-C₁₀cycloalkenyl or a C₆-C₁₄aryl; where the alkyl, alkenyl, cycloalkyl, cycloalkenyl, and aryl are optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷, C(O)OR⁷, C(O)NH₂, C(O)NHR⁷, C(O)NR⁷R⁷′, tetrahydrofuran-2-yl, tetrahydrofuran-3-yl, NO₂, OCF₃, and CF₃; provided that in formula (IA) at least one of R² and R³ is different than H;
or R² and R³ are bound to form a 5-or 6-membered cycloalkyl group containing the carbon atom bearing R² and the carbon atom bearing R³; where the 5-or 6-membered cycloalkyl is optionally substituted with one or more substituents selected from the group consisting of halogen, linear or branched C₁-C₆alkyl, linear or branched C₂-C₆alkenyl, C₃-C₁₀cycloalkyl, C₃C₁₀cycloalkenyl, linear or branched C₁-Csalkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
R⁴ represents H or a linear or branched C₁-C₁₈alkyl, C₃10cycloalkyl or C₆-C₁₀aryl;
R⁵ and R⁶, which are identical or different, represent a linear or branched C₁-C₁₈alkyl, linear or branched C₂-C₁₈alkenyl, C₃-C₁₀cycloalkyl, C₃-C₁₀cycloalkenyl or C₆-C₁₄aryl, where the alkyl, alkenyl, cycloalkyl, cycloalkenyl and aryl are optionally substituted with one or more substituents selected from the group consisting of halogen, linear or branched C₁-C6alkyl, linear or branched C₂-C₆alkenyl, C₃-C₁₀cycloalkyl, C₃-C₁₀cycloalkenyl, linear or branched C₁-C₆alkoxy, C₃C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O) NR⁷R⁷′, tetrahydrofuran-2-yl, tetrahydrofuran-3-yl, NO₂, CN, NH₂, OCF₃, and CF₃;
or R⁵ and R⁶ are bound to form a saturated or unsaturated 5-or 6-membered heterocyclic group containing the nitrogen atom bearing R⁶ and the carbon atom bearing R⁵ , where the 5-or 6-membered heterocyclic group optionally includes 1 or 2 additional heteroatoms selected from N, O and S, and where the 5-or 6-membered heterocyclic group is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C-₁₄aryl, NHR⁷, NR⁷R⁷, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃

or R⁵ and R⁶ are bound to form a saturated or partially unsaturated 7-to 10-membered fused heterocyclic group containing the nitrogen atom bearing R⁶ and the carbon atom bearing R⁵ where the 7-to 10-membered fused heterocyclic group optionally includes 1 or 2 additional heteroatoms selected from N, O and S, and where the 7-to 10-membered fused heterocyclic group is optionally substituted with one or more substituents selected from the group consisting of halogen, linear or branched C₁-C₆lkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷, C(O)OR⁷, C(O)NH₂, C(O)NHR⁷, C(O)NR⁷R⁷, tetrahydrofuran-2-yl, tetrahydrofuran-3-yl, NO₂, CN, NH₂, OCF₃, and CF₃;
R⁷and R⁷′, which are identical or different, represent a C₁-C₆alkyl, C₁-C₆ycloalkyl, C₂-C₆alkenyl, C₂-C₆cycloalkenyl, or C₆-C₁₄aryl.

In some optional aspects, the iron-based complex can be of formula (IA′) or (IB′)

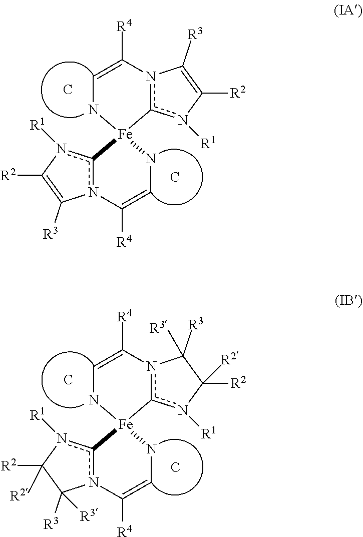

wherein

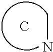

represents a moiety selected from the group consisting of

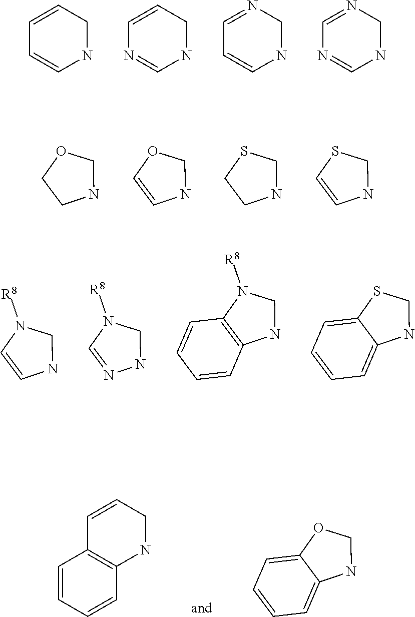

wherein R⁸ is a linear or branched C₁-C₆alkyl or C₆-C₁₄aryl and the moiety is optionally substituted with one or more substituents selected from the group consisting of halogen, linear or branched C₁-C₆alkyl, linear or branched C²-C⁶alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃, wherein R⁷ and R⁷′ are as defined herein.

According to another aspect, there is provided the use of the iron-based complex as defined herein, for catalyzing a hydrosilylation reaction.

According to yet another aspect, there is provided a method for preparing a silyl ether from a ketone or an aldehyde comprising:
hydrosilylation of the ketone or aldehyde in the presence of the iron-based complex as defined herein to form the silyl ether.

According to yet another aspect, there is provided a method for synthesizing an alcohol from a ketone or an aldehyde comprising:
hydrosilylation of the ketone or aldehyde in the presence of the iron-based complex as defined herein to form a silyl ether; and
hydrolyzing the silyl ether to obtain the alcohol.

According to a further aspect, there is provided a process for preparing the iron-based complex as defined herein, comprising
reacting a compound of formula (II)

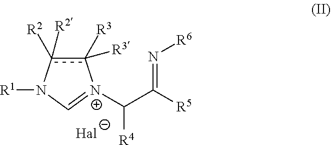

wherein
[symbol] represents a single bond or a double bond, and when [symbol] represents a double bond then R²′ and R³′ are absent;
R¹, R², R²′, R³, R³′, R⁵ , and R⁶ are as defined herein and R⁴ is H; with an iron salt.

Some aspects of the present technology will become readily apparent to those skilled in the art from the following detailed description, wherein various embodiments are described. The drawings and detailed description are to be regarded as illustrative in nature and not as restrictive.

### DESCRIPTION OF DRAWINGS

FIG. 1 represents the X-ray structure of iron-based complex [2]. Thermal ellipsoids are shown at 50% probability. All hydrogen atoms are omitted for clarity.

FIG. 2 represents the X-ray structure of iron-based complex [3]. Thermal ellipsoids are shown at 50% probability. All hydrogen atoms are omitted for clarity.

FIG. 3 represents the X-ray structure of iron-based complex [4]. Thermal ellipsoids are shown at 50% probability. All hydrogen atoms are omitted for clarity.

FIG. 4 represents the X-ray structure of iron-based complex [5]. Thermal ellipsoids are shown at 50% probability. All hydrogen atoms are omitted for clarity.

FIG. 5 represents the X-ray structure of iron-based complex [6]. Thermal ellipsoids are shown at 50% probability. All hydrogen atoms are omitted for clarity.

FIG. 6 represents the X-ray structure of iron-based complex [7]. Thermal ellipsoids are shown at 50% probability. All hydrogen atoms are omitted for clarity.

FIG. 7 represents the X-ray structure of iron-based complex [8]. Thermal ellipsoids are shown at 50% probability. All hydrogen atoms are omitted for clarity.

FIG. 8 represents the X-ray structure of iron-based complex [9]. Thermal ellipsoids are shown at 50% probability. All hydrogen atoms are omitted for clarity.

FIG. 9 represents the X-ray structure of iron-based complex [10]. Thermal ellipsoids are shown at 50% probability. All hydrogen atoms are omitted for clarity.

FIG. 10 represents the X-ray structure of iron-based complex [11]. Thermal ellipsoids are shown at 50% probability. All hydrogen atoms are omitted for clarity.

FIG. 11 represents the X-ray structure of iron-based complex [12]. Thermal ellipsoids are shown at 50% probability. All hydrogen atoms are omitted for clarity.

FIG. 12 represents the X-ray structure of iron-based complex [13]. Thermal ellipsoids are shown at 50% probability. All hydrogen atoms are omitted for clarity.

FIG. 13 represents the X-ray structure of iron-based complex [14]. Thermal ellipsoids are shown at 50% probability. All hydrogen atoms are omitted for clarity.

FIG. 14 represents the X-ray structure of iron-based complex [15]. Thermal ellipsoids are shown at 50% probability. All hydrogen atoms are omitted for clarity.

FIG. 15 represents the X-ray structure of iron-based complex [16]. Thermal ellipsoids are shown at 50% probability. All hydrogen atoms are omitted for clarity.

FIG. 16 represents the X-ray structure of iron-based complex [17]. Thermal ellipsoids are shown at 50% probability. All hydrogen atoms are omitted for clarity.

FIG. 17 represents the X-ray structure of iron-based complex [18]. Thermal ellipsoids are shown at 50% probability. All hydrogen atoms are omitted for clarity.

FIG. 18 represents the X-ray structure of iron-based complex [19]. Thermal ellipsoids are shown at 50% probability. All hydrogen atoms are omitted for clarity.

FIG. 19 represents the X-ray structure of iron-based complex [20]. Thermal ellipsoids are shown at 50% probability. All hydrogen atoms are omitted for clarity.

FIG. 20 represents the X-ray structure of iron-based complex [21]. Thermal ellipsoids are shown at 50% probability. All hydrogen atoms are omitted for clarity.

FIG. 21 represents the X-ray structure of iron-based complex [22]. Thermal ellipsoids are shown at 50% probability. All hydrogen atoms are omitted for clarity.

### DETAILED DESCRIPTION

### Definitions

All technical and scientific terms used herein have the same meaning as commonly understood by one ordinary skilled in the art to which the present technology pertains. For convenience, the meaning of certain terms and phrases used herein are provided below.

To the extent the definitions of terms in the publications, patents, and patent applications incorporated herein by reference are contrary to the definitions set forth in this specification, the definitions in this specification control. The section headings used herein are for organizational purposes only, and are not to be construed as limiting the subject matter disclosed.

The terminology used herein is for the purpose of describing particular embodiments only and is not intended to be limiting. It should be noted that, the singular forms “a”, “an”, and “the” include plural forms as well, unless the content clearly dictates otherwise. Thus, for example, reference to a composition containing “a compound” also contemplates a mixture of two or more compounds. It should also be noted that the term “or” is generally employed in its sense including “and/or” unless the content clearly dictates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the “iron-based complex” or “iron-based compound” and equivalent expressions refer to compounds described in the present application, e.g., those encompassed by structural Formulae IA, IB, IA′, IB′, optionally with reference to any of the applicable embodiments, and also includes exemplary compounds, for example, Compounds [5] to [14] mentioned herein, as well as their isomers and/or solvates when applicable.

Unless otherwise stated, structures depicted herein are also meant to include all isomeric (e.g., enantiomeric, diastereomeric, and geometric (or conformational)) forms of the structures; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the present description. Unless otherwise stated, all tautomeric forms of the compounds are within the scope of the present description.

Definitions of specific functional groups and chemical terms are described in more detail below.

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. Hydrogen atoms should be inferred to be part of the compound.

Abbreviations may also be used throughout the application, unless otherwise noted, such abbreviations are intended to have the meaning generally understood in the field. Examples of such abbreviations can include Me (methyl), Et (ethyl), Pr (propyl), iPr (isopropyl), Bu (butyl), tBu (tert-butyl), iBu (iso-butyl), sBu (sec-butyl), Ph (phenyl), Bn (benzyl), Cy (cyclohexyl), Mes (mesityl), Dipp (2,6-diisopropylphenyl).

The number of carbon atoms in a hydrocarbyl substituent can be indicated by the prefix “Cₓ-C_{y},” where x is the minimum and y is the maximum number of carbon atoms in the substituent. When reference is made to “x to y membered” heterocyclic ring (e.g., heterocycloalkyl or heteroaryl), then x and y define respectively, the minimum and maximum number of atoms in the cycle, including carbons as well as heteroatom(s).

The term “halogen” as used herein refer to an atom selected from fluorine (fluoro, —F), chlorine (chloro, —Cl), bromine (bromo, —Br), and iodine (iodo, —I).

The term “heteroatom” means one or more of oxygen, sulfur, or nitrogen, atoms.

The term “alkyl” as used herein, refers to a saturated, straight-(linear) or branched-chain hydrocarbon radical typically containing from 1 to 20 carbon atoms, or from 1 to 18 carbon atoms, or from 1 to 10 carbon atoms, or from 1 to 6 carbon atoms. For example, “C₁-C₆alkyl” contains from one to six carbon atoms. Examples of alkyl radicals include, but are not limited to, methyl, ethyl, propyl, isopropyl, n-butyl, sec-butyl, tert-butyl, neopentyl, n-hexyl, heptyl, octyl radicals and the like.

The term “alkenyl” as used herein, denotes a linear-or branched-chain hydrocarbon radical containing one or more double bonds and typically from 2 to 20 carbon atoms, or from 1 to 18 carbon atoms, or from 1 to 10 carbon atoms, or from 1 to 6 carbon atoms. The double bond(s) in the alkenyl group can be at any place on the hydrocarbon chain. For example, “C₂-C₆ alkenyl” contains from two to six carbon atoms. Alkenyl groups include, but are not limited to, for example, ethenyl, propenyl, butenyl, 1-methyl-2-buten-1-yl, heptenyl, octenyl and the like.

The term “alkoxy” as used herein, refers to group-Oalkyl, wherein alkyl is as defined herein. For example, the alkoxy group may be —O(C₁-C₆alkyl). Examples of alkoxy groups include, without being limited to, -OMe,-OEt,-OiPr, etc.

The term “cycloalkyl” refers to a group comprising a saturated carbocyclic ring in a monocyclic or polycyclic ring system, including spiro (sharing one atom), fused (sharing at least one bond) or bridged (sharing two or more bonds) carbocyclic ring systems, having from three to ten ring members. The term “C₃-Cₙcycloalkyl” refers to a cycloalkyl group having from 3 to the indicated “n” number of carbon atoms in the ring structure. The term “cycloalkenyl” refers to partially unsaturated carbocyclic rings in a monocyclic or polycyclic ring system, including spiro, fused or bridged carbocyclic ring systems, having from three to ten ring members. The term “C₃-Cₙcycloalkenyl” refers to a cycloalkenyl group having from 3 to the indicated “n” number of carbon atoms in the ring structure. Examples of cycloalkyl or cycloalkenyl groups can include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo [4,3,0] nonanyl, norbornyl, 1-adamantly and 2-adamantyl, and the like.

As used herein, the terms “heterocycloalkyl” and “heterocyclic group” are used interchangeably and refer to a chemically stable 3-to 7-membered monocyclic or 7-to 10-membered bicyclic heterocyclic moiety that is either saturated or partially unsaturated, and include, in addition to carbon atoms, one or more, preferably one to four, heteroatoms, as defined above. The 3-to 7-membered monocyclic heterocyclic group includes 3, 4, 5, 6, or 7 atoms in total in the ring, by counting carbon atoms and heteroatoms. The 7-to 10-membered bicyclic heterocyclic moiety includes a fused carbocyclic ring system. When referring to “7-to 10-membered”, one means that the fused heterocyclic moiety includes 7, 8, 9 or 10 atoms in total for the two fused rings, by counting carbon atoms and heteroatoms. As used herein, the expression “fused heterocyclic group” thus refers to a heterocyclic group including two fused rings. A heterocyclic group can be attached to its pendant group at any heteroatom or carbon atom that results in a chemically stable structure and any of the ring atoms can be optionally substituted.

The term “aryl” used herein refers to a monocyclic moiety or to a bicyclic or tricyclic fused ring system having a total of six to 14 ring members, wherein at least one ring in the system is aromatic and wherein each ring in the system contains three to seven ring members. The term “C₆-Cₙaryl” refers to an aryl ring having from 6 to the indicated “n” number of carbon atoms in the ring structure. In certain embodiments of the present description, “aryl” refers to an aromatic ring system which includes, but not limited to, phenyl, biphenyl, naphthyl, azulenyl, anthracyl and the like, which may bear one or more substituents.

The term “heteroaryl” used alone or as part of a larger moiety, refers to groups having 5 to 18 ring atoms, preferably 6 to 10 ring atoms, e. g., 5, 6, or 9 ring atoms, having, in addition to carbon atoms, from one to five heteroatoms. The term “heteroatom” includes but is not limited to nitrogen, oxygen, or sulfur, and includes any oxidized form of nitrogen or sulfur, and any quaternized form of a basic nitrogen. A heteroaryl may be a single ring, or two or more fused rings. A heteroaryl group can be attached to its pendant group at any heteroatom or carbon atom that results in a chemically stable structure. A heteroaryl group may be mono-or bicyclic. Heteroaryl groups include rings that are optionally substituted.

As described herein, compounds of the present description can be optionally substituted. In general, the term “substituted” means that one or more hydrogens of the designated moiety are replaced with a suitable substituent. Unless otherwise indicated, a substituted group may have a suitable substituent at each substitutable position of the group, and when more than one position in any given structure may be substituted with more than one substituent selected from a specified group, the substituent may be either the same or different at each position. Combinations of substituents envisioned under the present description are preferably those that result in the formation of chemically stable or chemically feasible compounds. The term “chemically stable”, as used herein, refers to compounds that are not substantially altered when subjected to conditions to allow for their production, detection, and, in certain embodiments, their recovery, purification, and use for one or more of the purposes disclosed herein.

### Iron-Based Complexes

The present application provides new iron-based complexes formula (IA) or (IB)

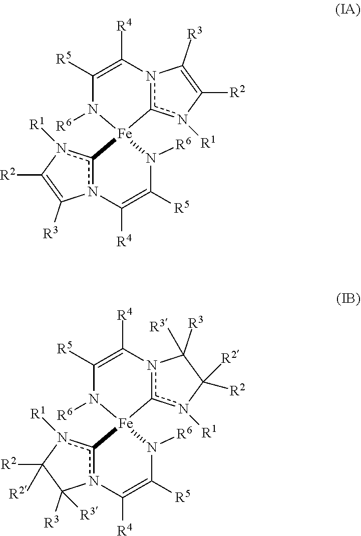

wherein:
the chain

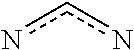

comprise one single bond and one double bond;
R¹ represents a linear or branched C₁-C₁₈alkyl, linear or branched C₂-C₁₈alkenyl, C₃-C₁₀cycloalkyl, C₃-C₁₀cycloalkenyl or C₆-C₁₄aryl, where the alkyl, alkenyl, cycloalkyl, cycloalkenyl and aryl are optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
R², R²′, R³ and R³′, which are identical or different, represent H, a linear or branched C₁-C₁₀alkyl, linear or branched C₂-C₁₀alkenyl, C₃-C₁₀cycloalkyl, C₃-C₁₀cycloalkenyl or a C₆-C₁₄aryl; where the alkyl, alkenyl, cycloalkyl, cycloalkenyl, and aryl are optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃; provided that in formula (IA) at least one of R² and R³ is different than H;
or R² and R³ are bound to form a 5-or 6-membered cycloalkyl group containing the carbon atom bearing R² and the carbon atom bearing R³; where the 5-or 6-membered cycloalkyl is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
R⁴ represents H or a linear or branched C₁-C₁₈alkyl, C₃-C₆cycloalkyl or C₆-C₁₀aryl;
R⁵ and R⁶ , which are identical or different, represent a linear or branched C₁-C₁₈alkyl, linear or branched C₂-C₁₈alkenyl, C₃-C₁₀cycloalkyl, C₃-C₁₀cycloalkenyl or C₆-C₁₄aryl, where the alkyl, alkenyl, cycloalkyl, cycloalkenyl and aryl are optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
or R⁵ and R⁶ are bound to form a saturated or unsaturated 5-or 6-membered heterocyclic group containing the nitrogen atom bearing R⁶ and the carbon atom bearing R⁵, where the 5-or 6-membered heterocyclic group optionally includes 1 or 2 additional heteroatoms selected from N, O and S, and where the 5-or 6-membered heterocyclic group is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C-₁₄aryl, NHR⁷, NR⁷R⁷), C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
or R⁵ and R⁶ are bound to form a saturated or partially unsaturated 7-to 10-membered fused heterocyclic group containing the nitrogen atom bearing R⁶ and the carbon atom bearing R⁵ where the 7-to 10-membered fused heterocyclic group optionally includes 1 or 2 additional heteroatoms selected from N, O and S, and where the 7-to 10-membered fused heterocyclic group is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
R⁷ and R⁷′, which are identical or different, represent a C₁-C₆alkyl, C₁-C₆cycloalkyl, C₂-C₆alkenyl, C₂-C₆cycloalkenyl, or C₆-C₁₄aryl.

In some embodiments, the groups R² and R³, which can be identical or different, can represent H, a linear or branched C₁-C₆alkyl, or a C₆aryl, where the alkyl and aryl are optionally substituted with one or more substituents selected from the group consisting of halogen, linear or branched C₁-C₆alkyl, C₆-C₁₀aryl, vinyl, NO₂, and CF₃, provided that in the formula (IA) at least one of R² and R³ is different than H.

In some embodiments, the groups R² and R³, which can be identical or different, can represent H, Me, Et, or Ph, provided that in the formula (IA) at least one of R² and R³ is different than H.

In further embodiments, R² can be H and R³ can represent a linear or branched C₁-C₆alkyl or a C₆aryl, where the alkyl and aryl are optionally substituted with one or more substituents selected from the group consisting of halogen, linear or branched C₁-C₆alkyl, C₆-C₁₀aryl, linear or branched C₁-C₆alkoxy, OCF₃, and CF₃. In some embodiments, R² can be H and R³ is H, Me, Et, or Ph.

In yet another embodiment, the groups R² and R³ can be identical and represent a linear or branched C₁-C₆alkyl, or a C₆aryl, where the alkyl and aryl are optionally substituted with one or more substituents selected from the group consisting of halogen, linear or branched C₁-C₆alkyl, C₆-C₁₀aryl, linear or branched C₂-C₆alkenyl, linear or branched C₁-C₆alkoxy, OCF₃ and CF₃.

In yet another embodiment, the groups R² and R³ can be identical and represent Me, Et, or Ph.

In other embodiments, the groups R² and R³ can be bound to form a 5-or 6-membered cycloalkyl group optionally substituted with 1 to 4 substituents selected from the group consisting of halogen, linear or branched C₁-C₆alkyl, C₆-C₁₀aryl, linear or branched C₂-C₆ alkenyl, linear or branched C₁-C₆alkoxy, OCF₃, and CF₃.

In some embodiments, the iron-based complex is of formula (IA)

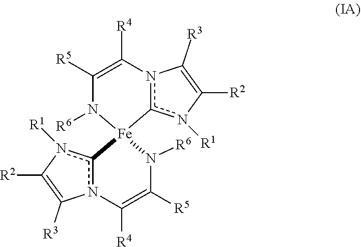

namely an iron-based compound where the carbon atom bearing the R² group and the carbon atom bearing the R³ groups are bound through a double bound. The groups R² and R³ can have any of the above-mentioned definitions and at least one of R² and R³ is different than H.

In some embodiments, the iron-based complex is of formula (IB)

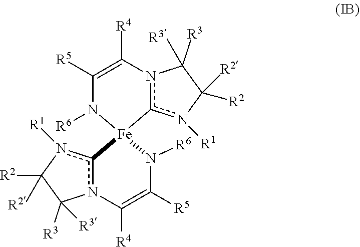

namely an iron-based compound where the carbon atom bearing the R² and R²′ groups and the carbon atom bearing the R³ and R³′ groups are bound through a single bound. In compound (IB), the groups R², R²′, R³ and R³′ can all represent H or each of R², R²′, R³ and R³′ can independently have the definitions mentioned above.

In some embodiments, the iron-based compound of formula (IB) is such that R¹, R², R³, R⁴, R⁵ and R⁶ can have any of the above-mentioned definitions, and R²′ and R³′ are identical or different and represent H, a linear or branched C₁-C₆alkyl or a C₆aryl, where the alkyl and aryl are optionally substituted with one or more substituents selected from the group consisting of halogen, linear or branched C₁-C₆alkyl, C₆-C₁₀aryl, linear or branched C₂-C₆alkenyl, linear or branched C₁-C₆alkoxy, OCF₃, and CF₃.

In some embodiments, the iron-based compound of formula (IB) is such that R²′ and R³′ are identical or different and represent H, Me, Et, or Ph.

In further embodiments, the iron-based compound of formula (IB) is characterized in that R²′ and R³′ are identical and represent H, Me, Et, or Ph.

In yet another embodiment, the iron-based complex is of formula (IB) where R² and R³ are identical and represent Me, Et, or Ph, and R²′ and R³′ are H.

In yet another embodiment, the iron-based complex is of formula (IB) where R²′ and R³′ represent H and R² and R³ are bound to form a 5-or 6-membered cycloalkyl group optionally substituted with 1 to 4 substituents selected from the group consisting of halogen, linear or branched C₁-C₆alkyl, C₆-C₁₀aryl, linear or branched C₂-C₆alkenyl, linear or branched C₁-C₆alkoxy, OCF₃, and CF₃. In a further embodiment, R²′ and R³′ can represent H and R² and R³ are bound to form a cyclohexyl group.

In some embodiments, the iron-based complex, being of formula (IA) or (IB), can include a group R¹ which represents a linear or branched C₁-C₆alkyl, a C₃-C₆cycloalkyl or C₆aryl, where the C₃-C₆cycloalkyl and C₆aryl are optionally substituted with one or more substituents selected from the group consisting of halogen, linear or branched C₁-C₆alkyl, C₆-C₁₀aryl, linear or branched C₂-C₆alkenyl, linear or branched C₁-C₆alkoxy, OCF₃, and CF₃.

In other embodiments, the group R¹ in formula (IA) or (IB) can represent a linear or branched C₁-C₆alkyl, a C₃-C₆cycloalkyl or C₆aryl, where the C₃-C₆cycloalkyl and C₆aryl are optionally substituted with 1 to 3 linear or branched C₁-C₆alkyl.

In yet another embodiment, the group R¹ can represent a linear or branched C₁-C₆alkyl, or C₆aryl, where the C₆aryl is optionally substituted with 1 to 3 linear or branched C₁-C₄alkyl.

Hence, some examples of R¹ group can include Me, Et, nPr, iPr, nBu, sBu, tBu, C₆H₁₁, Ph, Bn, C₆H₄Me, C₆H₃(Me)₂, C₆H₂(Me)₃, C₆H₄(iPr), C₆H₃(iPr)₂, C₆H₂(iPr)₃, C₆H₄(tBu), C₆H₃(tBu)₂, or C₆H₂(tBu)₃. In some preferred embodiments, R¹ can represent Et, Ph, tBu, C₆H₂(Me)₃, or C₆H₃(iPr)₂.

In other embodiments, the group R⁴ in formula (IA) or (IB) can represent H or a linear or branched C₁-C₄alkyl. For instance, R⁴ can represent H or Me. In some preferred embodiments, R⁴ can represent H.

In some embodiments, the iron-based complex of formula (IA) or (IB), can include R⁵ and R⁶, which are identical or different, and represent a linear or branched C₁-C₆alkyl or C₆-aryl, where the alkyl and aryl are optionally substituted with one or more substituents selected from the group consisting of halogen, linear or branched C₁-C₆alkyl.

In some embodiments, the iron-based complex of formula (IA) or (IB), can include R⁵ and R⁶, which are identical or different, and represent an alkyl group being Me, Et, nPr, iPr, nBu, sBu, tBu or C₆-aryl, where the aryl is optionally substituted with one or more substituents selected from the group consisting of linear or branched C₁-C₆alkyl, such as Me, Et, nPr, iPr, nBu, sBu, or tBu. In some embodiments, R⁵ can be methyl and R⁶ can be C₆H₂(Me)₃.

In some embodiments, the iron-based complex of formula (IA) or (IB), can include R⁵ and R⁶ which are bound to form a saturated or unsaturated 5-or 6-membered heterocyclic group containing the nitrogen atom bearing R⁶ and the carbon atom bearing R⁵, where the 5-or 6-membered heterocyclic group optionally includes 1 or 2 additional heteroatoms selected from N, O and S, and where the 5-or 6-membered heterocyclic group is optionally substituted with 1 to 4 substituents selected from the group consisting of halogen, linear or branched C₁-C₆alkyl, C₆-C₁₀aryl, linear or branched C₂-C₆alkenyl, linear or branched C₁-C₆alkoxy, OCF₃ and CF₃.

In another embodiment, R⁵ and R⁶ can be bound to form a saturated or unsaturated 5-or 6-membered heterocyclic group containing the nitrogen atom bearing R⁶ and the carbon atom bearing R⁵, where the 5-or 6-membered heterocyclic group optionally includes 1 or 2 additional heteroatoms selected from N, O and S, and where the 5-or 6-membered heterocyclic group is optionally substituted with a linear or branched C₁-C₆alkyl.

In yet another embodiment, R⁵ and R⁶ can be bound to form a saturated or partially unsaturated 9-or 10-membered fused heterocyclic group containing the nitrogen atom bearing R⁶ and the carbon atom bearing R⁵ , where the 9-or 10-membered fused heterocyclic group optionally includes 1 or 2 additional heteroatoms selected from N, O and S, and where the 9-or 10-membered fused heterocyclic group is optionally substituted with a linear or branched C₁-C₆alkyl.

In yet another embodiment, R⁵ and R⁶ can be bound to form a partially unsaturated 9-or 10-membered fused heterocyclic group containing the nitrogen atom bearing R⁶ and the carbon atom bearing R⁵, where the 9-or 10-membered fused heterocyclic group includes 1 or 2 additional heteroatoms selected from N and O, and where the 9-or 10-membered fused heterocyclic group is optionally substituted with a linear or branched C₁-C₆alkyl.

In some embodiments, the iron-based complex can have the formula (IA′) or (IB′) below:

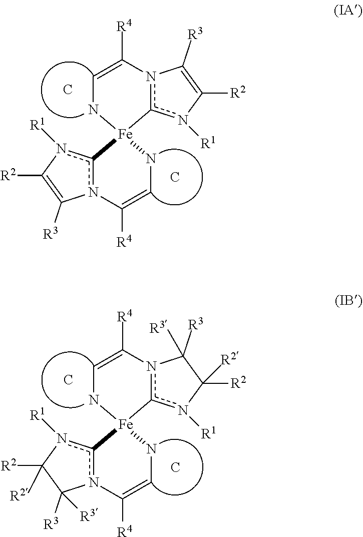

wherein

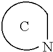

represents a moiety selected from the group consisting of

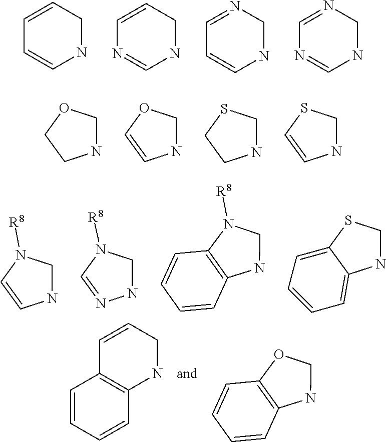

wherein R⁸ is a linear or branched C₁-C₆alkyl or C₆-C₁₄aryl and the moiety is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃, where R⁷ and R⁷′ are as defined above.

In some embodiments, the moiety

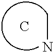

as described above, can be substituted with a linear or branched C₁-C₆ alkyl. In some embodiments, the moiety

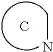

is unsubstituted.

Examples of iron-based complexes according to the present disclosure can include:

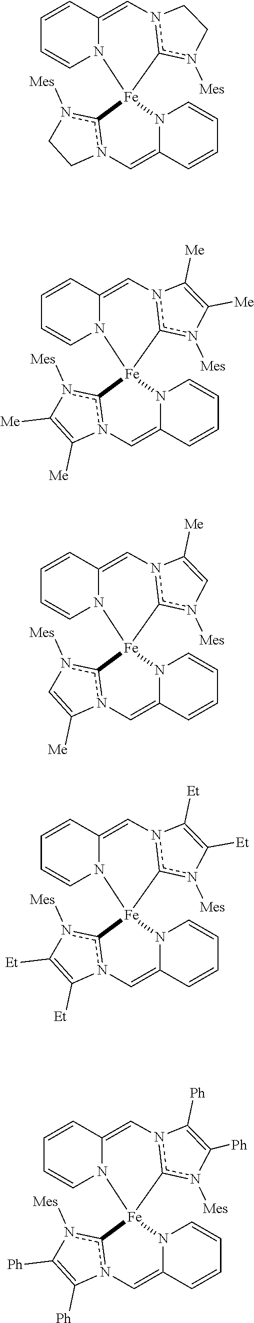

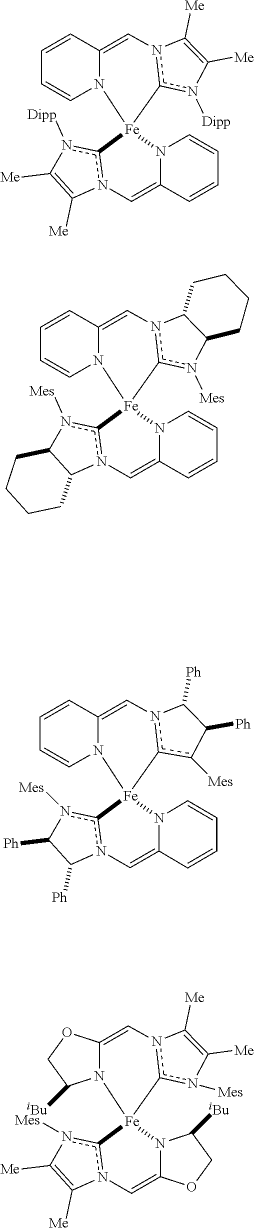

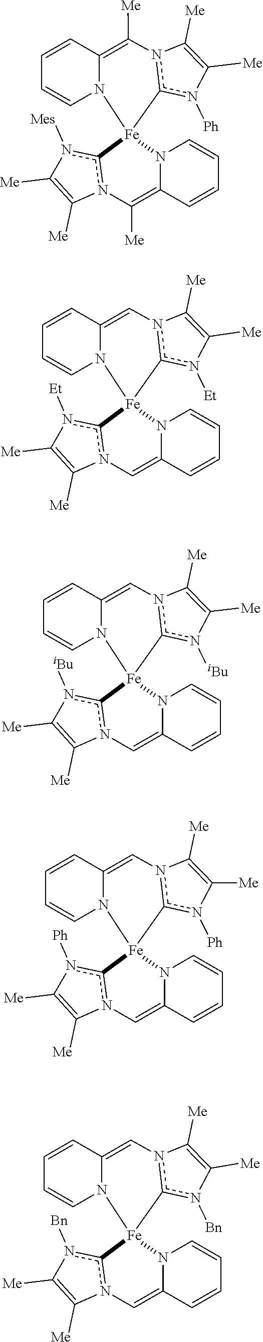

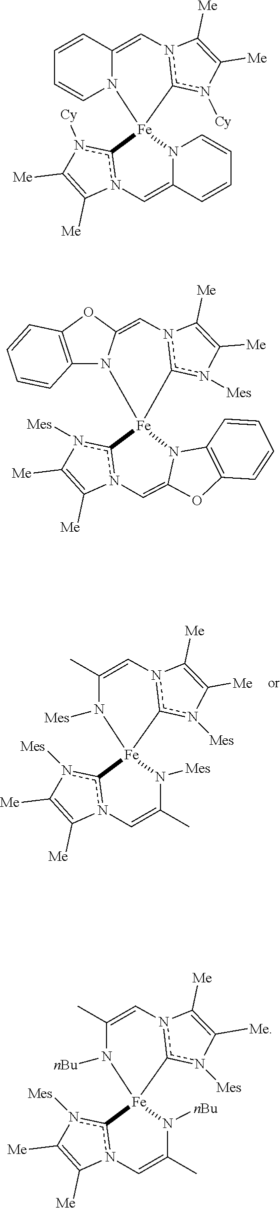

In some particular embodiments, the iron-based complex can include one of the following complexes:

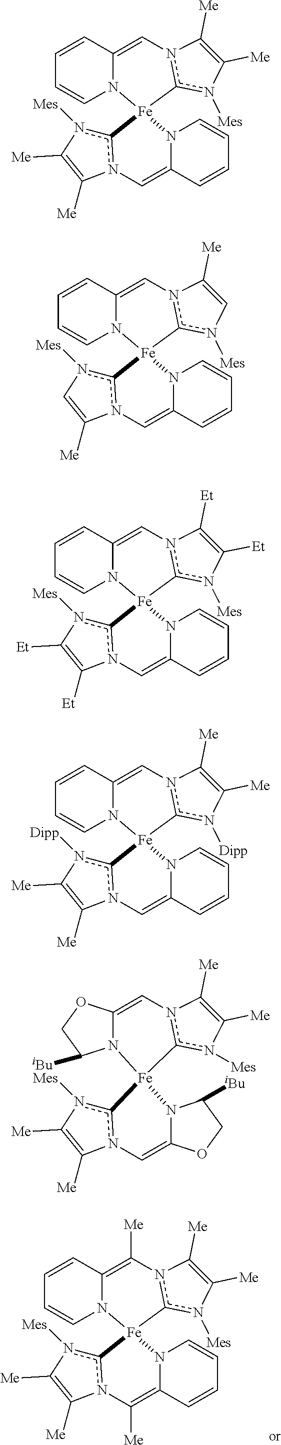

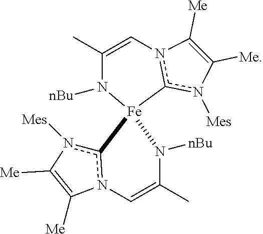

In some embodiments, the iron-based complex can be protected with a wax to prepare a wax-protected iron-based complex. By dispersing the iron-based complex within a wax, one can protect the complex from air and the so-obtained protected catalyst can be stored for months and handled in ambient air. In some embodiments, the wax used to protect the iron-based complex can be a paraffin wax such as a paraffin wax having a melting temperature in the range from about 55° C. to about 60° C.

### Use of the Iron-Based Complexes in Hydrosilylation Reactions

The iron-based complexes as disclosed herein are particularly useful for catalyzing hydrosilylation reactions, more particularly ketones or aldehydes hydrosilylation. Certain iron-based complexes that are disclosed herein can display higher activities than iron-based complex [1] mentioned herein, making them efficient catalysts. The inventors noted that, in some embodiments, structural modification such as the introduction of certain substituents on the backbone of the N-heterocyclic carbene (NHC) ring (groups R², R²′, R³, and/or R³′) in the complexes, can allow increasing their catalytic efficiency towards hydrosilylation compared to complex [1]. More particularly, an increase in catalytic activity was surprisingly observed for certain complexes where alkyl groups were added as substituents on the backbone of the NHC ring, although such a subtle modification usually does not impact the reactivity of the complexes. A general scope shows that the activity can be maintained over a range of aromatic and aliphatic ketones, even the extremely challenging ones.

Hydrosilylation of ketones and aldehydes is generally performed using hydrosilanes such as PhSiH₃, Me(OEt)₂SiH, (EtO)₃SiH, Ph₂SiH₂, Et₃SiH. Other possible hydrosilanes can include polymethylhydrosiloxane (PMHS) and 1,1,3,3-tetramethyldisiloxane (TMDS). In some embodiments, the iron-based complexes of the present disclosure can be used for catalyzing hydrosilylation of ketones and aldehydes in the presence of a hydrosilane selected from the group consisting of PhSiH₃, Me (OEt)₂SiH, (EtO)₃SiH, Ph₂SiH₂, Et₃SiH, polymethylhydrosiloxane (PMHS), 1,1,3,3-tetramethyldisiloxane (TMDS), and any mixture thereof. In preferred embodiments, PMHS and/or TMDS are advantageously used since they are cheaper than the other above-mentioned hydrosilanes. PMHS can preferably be used in certain embodiments.

In some embodiments, the hydrosilylation reaction that is performed in the presence of the iron-based complex as catalyst, does not require any heating and can advantageously be carried out at ambient temperature. By “ambient temperature”, one means the temperature of the room/place where the reaction is performed and can usually range between about 15° C. and about 30° C., or between about 18° C. and about 25° C., or between about 20° C. and about 25° C. If required, the hydrosilylation could also be performed under heating or cooling.

In some embodiments, the hydrosilylation reaction can be performed using a solvent, particularly when the ketone or aldehyde is a solid at ambient temperature. However, when the ketone or aldehyde is liquid at ambient temperature, an additional solvent may not be required. As mentioned above, PMHS and TMDS can be preferably used in certain embodiments, and since these two silanes are cheap enough, they can also serve as solvent for the hydrosilylation reaction involving a liquid ketone. Hence, using PMHS and TMDS can be both economically and environmentally advantageous. However, in some embodiments, it may be required to use other silanes such as PhSiH3, Me (OEt)₂SiH, (EtO)₃SiH, Ph₂SiH₂, Et₃SiH, which are more expensive. Although such silanes could be used without using a solvent for the hydrosilyation reaction, it may be preferable to use them in combination with a solvent for economical reasons, even when the ketone or aldehyde is liquid. Solvents that can be used for hydrosilylation reactions are well known in the field.

The iron-based complexes of the present disclosure can be used to catalyze the hydrosilylation of various types of ketones or aldehydes, including sterically encumbered ketones such as sterically encumbered aliphatic or aromatic ketones. In some embodiments, the iron-based complexes can catalyze the hydrosilylation of ketones of formula R(CO)R′ or aldehydes of formula RCHO, where:
R and R′ are independently selected from the group consisting of linear or branched C₁-C₁₀alkyl, linear or branched C₂-C₁₀alkylenyl, C₃-C₁₀cycloalkyl, 4-to 6-membered heterocycloalkyl, C₆-C₁₀aryl, and 6 to 10-membered heteroaryl; or
in the ketone R(CO)R′, R and R′ can form with the carbon atom of the CO group, a saturated or unsaturated C₄-C₁₀cycloalkyl or 5-to 10-membered heterocycloalkyl, and
the alkyl, cycloalkyl, heterocycloalkyl, aryl and heteroaryl are optionally substituted with one or more substituents selected from the group consisting of halogen, linear or branched C₁-C₆alkyl, linear or branched C₁-C₆alkoxy, NH₂, CN, and NO₂.

In some embodiments, the ketones and aldehydes that can be hydrosilylated using the iron-based complexes can be of formula R(CO)R′ or formula RCHO respectively, where R and R′ independently represent Me, Et, iPr, tBu, cyclopropyl, C₆H₅, C₆H₄F, C₆H₄Cl, C₆H₄Br, C₆H₄I, C₆H₄Me, C₆H₄OMe, C₆H₄NH₂, C₆H₄NO₂, C₆H₂Me₃, or R and R′ form together with the carbon to which they are attached a group C₆H₁₀.

In certain embodiments, the amount of iron-based complex required for the hydrosilylation reaction can be quite low, which can be an advantage for large scale industrial applications, not only because this implies reduced costs for the chemical materials themselves but also because this could allow reduced management operation for recovering and/or eliminating the catalyst after the reaction. In some embodiments, an iron-based complex loading as low as 0.1 mol % based on the molar concentration of the ketone or aldehyde, is sufficient for catalyzing the hydrosilylation reaction. The iron-based complex loading can even be less than 0.1 mol % or less than about 0.05 mol % in some embodiments. In some embodiments, the catalyst loading can even be in the ppm range. For instance, a catalyst loading as low as about 12.5 ppm (i.e., 1 mol of catalyst and 80000 mol of ketone) can be sufficient for performing the hydrosilylation.

Although examples of ketones and aldehydes that can be hydrosilylated using the iron-based catalysts of the present disclosure have been mentioned above, there are not limited to these examples. The iron-based catalysts of the present disclosure can be useful to hydrosilylate a large variety of ketones and aldehydes. Once hydrosilylated, the corresponding silyl ethers of these ketones and aldehydes can then be hydrolyzed to form alcohols, as will be detailed below.

### Synthesis of Silyl Ether Intermediates and Alcohols

As mentioned above, the present iron-based complexes can catalyze the hydrosilylation of ketones and aldehydes to form the corresponding silyl ethers, which then can themselves be hydrolyzed to form alcohols.

Therefore, the present disclosure further relates to a method for preparing a silyl ether from a ketone or an aldehyde, wherein the method involves the hydrosilylation of the ketone or aldehyde in the presence of the iron-based complex as defined herein to form the corresponding silyl ether.

The present disclosure also relates to a method for synthesizing an alcohol from a ketone or an aldehyde, wherein the method first involves the hydrosilylation of the ketone or aldehyde in the presence of the iron-based complex as defined herein to form the corresponding silyl ether, and then the hydrolysis of the silyl ether to obtain the alcohol.

The hydrosilylation rection can be performed using any hydrosilane known in the art. In some embodiments, the hydrosilane can be any one of the previously mentioned hydrosilanes, and in preferred embodiments, polymethylhydrosiloxane (PMHS) or 1,1,3,3-tetramethyldisiloxane (TMDS).

As discussed above, the hydrosilylation can be performed at ambient temperature, with or without the use of a solvent. When, the hydrosilylation involves a liquid ketone or aldehyde and the use of PMHS or TMDS, an additional solvent is generally not necessary. When the ketone or aldehyde is solid at ambient temperature, a solvent is generally required even when using PMHS and TMDS as hydrosilane.

The silyl ether intermediates can be prepared from any type of ketones or aldehydes and such silyl ether intermediates can thus afford a large variety of alcohols. Examples of ketones and aldehydes that can be used for the synthesis of the silyl ether intermediates are mentioned above but are not limited to such examples.

Alcohols can be prepared from the silyl ether intermediates through a further hydrolyzing step, which, in some embodiments, can be performed under basic conditions, e.g., using aqueous solutions of sodium or potassium hydroxide. The hydrolysis step can be performed by conventional methods known in the field.

The present methods for preparing the silyl ethers and the corresponding alcohols using the iron-based complexes of the present disclosure can be performed starting from various ketones and aldehydes, even including sterically encumbered ketones, which may be more difficult to reduce under conventional methods. A further advantage of the present methods is that the iron-based catalysts can present a good activity even at low loading and can be used in combination with low cost hydrosilanes. Furthermore, in some embodiments the iron-based catalysts used in the present methods can be easily eliminated after the hydrolysis. The hydrosilane (e.g., PMHS) will be transformed into silica gel after hydrolysis and the insoluble inactive catalyst will adsorb onto silica and be lost during the extraction step of the final product isolation. In alternative embodiments, the iron-based catalyst can be grafted onto a solid-state support, which can allow for its recovery at the end of the process.

### Preparation of the Iron-Based Complexes

The iron-based complexes and any intermediates thereof according to the present application may be prepared by as will be explained below and as exemplified in the Examples. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired products of the present description. Synthetic chemistry transformations useful in synthesizing the compounds described herein are known in the art. The synthesized compounds can be separated from a reaction mixture and further purified by standard methods such as column chromatography, high pressure liquid chromatography, or recrystallization.

In one embodiment, the process for preparing the iron-based complexes of the present disclosure can include reacting a compound of formula (II) below with an iron salt

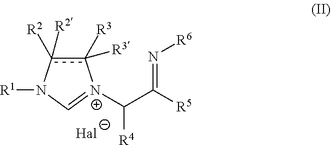

wherein in formula (II):
[symbol] represents a single bond or a double bond, and when [symbol] represents a double bond then R²′ and R³′ are absent; R⁴ is H and R¹, R², R²′, R³, R³′, R⁵, and R⁶ are as defined herein.

The reaction between the compound of formula (II) and the iron salt can be performed in the presence of a base. In some embodiments, the base can be selected from the group consisting of BuLi, PhLi, Lithium diisopropylamide (LDA), KBn, NaH, KH, Lithium HexaMethylDiSilazide (LiHMDS), and Potassium HexaMethylDiSilazide (KHMDS). However, other types of bases can be used and one skilled in the art could select a suitable base for the reaction.

Various iron salts can be used for preparing the iron-based complexes and one skilled in the art will be able to choose a proper salt. In some embodiments, the iron salt can be selected from the group consisting of FeCl₂, FeBr₂, Fe(HMDS)₂, Fe(OAc)₂, Fe(OTf)₂, and FeCl₂(THF)_{1.5}. In some preferred embodiments, FeCl₂(THF)_{1.5} can be used as the iron salt.

In some embodiments, the reaction between the iron salt and the ligand precursor of formula (II) can be performed at ambient temperature and in the presence of a solvent. One skill in the field can select a suitable solvent for performing the complexation reaction. Examples of solvents that can be used ca include toluene, THF, Et₂O, dioxane, benzene, DME, xylene, mesitylene, or any mixture thereof.

In certain embodiments, where R⁵ and R⁶ are bound to form a saturated or unsaturated 5-or 6-membered heterocyclic group, as explained above, the compound of formula (II) can be obtained by reacting a compound of formula (III)

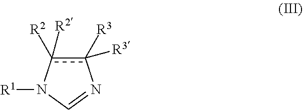

wherein
[symbol] represents a single bond or a double bond, and when [symbol] represents a double bond then R²′ and R³′ are absent;
and R¹, R², R²′, R³ and R³′ are as defined herein;
with a compound of formula (IV)

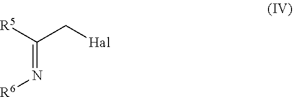

wherein R⁵ and R⁶ form together with the carbon and nitrogen atoms to which they are respectively attached, the saturated or unsaturated 5-or 6-membered heterocyclic group.

In some embodiments, the reaction between the compound of formula (III) and (IV) can be performed under heating, and can be performed in the presence of a solvent. In some embodiments, the reaction between the compound of formula (III) and (IV) is performed in the presence of a solvent under reflux. Various possible solvents can be contemplated, and examples include THF, ether, toluene, benzene, DME, and any mixture thereof. However, other solvents could be used.

In some implementations, the compound of formula (IV) can be a compound of formula (IVa)

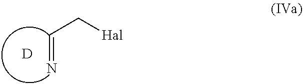

wherein

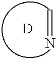

represents a moiety selected from the group consisting of

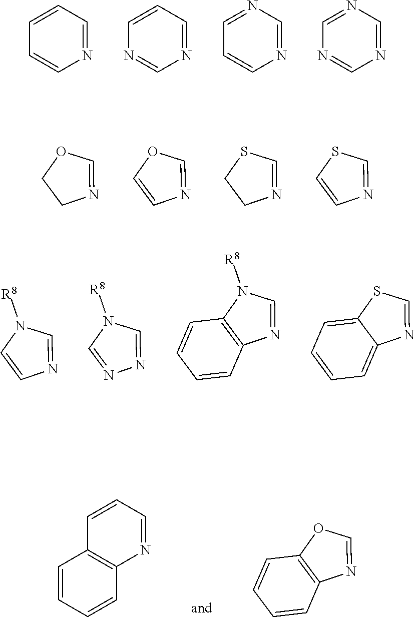

wherein R⁸ is as defined above.

the moiety

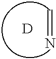

as described above, can be substituted with a linear or branched C₁-C₆alkyl. In some embodiments, the moiety

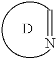

is unsubstituted.

In further embodiments, the iron-based complex where R⁴ represents a linear or branched C₁-C₁₈alkyl, or C₃-C₆cycloalkyl can be prepared by first synthesizing the iron-based complex where R⁴ is H, according to the above-described embodiments, then reacting the iron-based complex where R⁴ is H with a compound of formula R⁹-LG where LG is a leaving group and R⁹ is a linear or branched C₁-C₁₈alkyl, or C₃-C₆cycloalkyl, and then with a base.

The leaving group LG can be any group known in the field, and, in some embodiments, can include OTf, halogen, OTs, etc. In some embodiments, the base can be selected from the group consisting of BuLi, PhLi, Lithium diisopropylamide (LDA), KBn, NaH, KH, and alkali metal bis (trimethylsilyl) amide. The alkali metal bis (trimethylsilyl) amide can be Lithium bis (trimethylsilyl) amide (LiHMDS), or Potassium bis (trimethylsilyl) amide (KHMDS). The reaction to form the iron-based complex where R⁴ represents a linear or branched C₁-C₁₈alkyl, or C₃-C₆cycloalkyl, from the iron-based complex where R⁴ is H, can be performed in any suitable solvent, such as toluene, THF, ether, benzene, DME, or any mixture thereof. The reaction can be performed at room temperature.

A large variety of iron-based complexes can therefore be prepared under relatively simple reaction procedures. The resulting iron-based complexes have been shown to possess catalytic activity towards hydrosilylation reactions, which can make them attractive for industrial synthesis of alcohols involving the hydrosilylation of ketones or aldehydes. However, the present iron-based complexes can also be useful to prepare silyl ether intermediates that are desirable for other applications, such as for generating protected alcohols. For instance, the iron-based complexes can be used to catalyze the transformation of ketones or aldehydes into the protected hydroxyl function groups as silyl ethers in certain steps of multistep syntheses that use reagents that are incompatible with hydroxyl groups, e.g., Grignard and organolithium reagents.

### EXAMPLES

The Examples set forth herein below provide syntheses and experimental results obtained for certain exemplary compounds. Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such.

The following is to be construed as merely illustrative, and not limitations of the preceding disclosure in any way whatsoever. Those skilled in the art will promptly recognize appropriate variations from the procedures both as to reactants and as to reaction conditions and techniques. In some cases, starting materials or intermediates may be commercially available.

### Experimental

All reactions were carried out in a dinitrogen-filled glovebox or using the standard Schlenk techniques under dinitrogen. Glassware was dried in a 180° C. oven overnight. Diethyl ether, hexanes and pentane solvent were dried by refluxing and distilling over sodium under dinitrogen. THF and toluene solvent were dried by refluxing and distilling over sodium benzophenone ketyl under dinitrogen. C₆D₆, CD₂Cl₂, THF-d₈ and toluene-d₈ were degassed through three consecutive freeze-pump-thaw cycles. All solvents were stored over 3 Å molecular sieves prior to use. Unless otherwise noted, all NMR spectra were recorded at 25° C. on an Agilent DD2 600 MHz spectrometer or an Agilent DD2 500 MHZ spectrometer with ¹³C-sensitive cryogenically cooled probe. Chemical shifts are referenced to the solvent signals. The NMR signal assignments were made based on ¹H-COSY, ¹H-¹³C-HSQC, and ¹H-¹³C-HMBC NMR spectroscopy. Elemental analyses were carried out at the ANALEST at the University of Toronto. Unless otherwise noted, all chemicals were purchased from commercial sources and used as received.

### Syntheses of Ligand Precursors

Various ligand precursors were prepared to then be used in the synthesis of the iron complexes.

The chemical structure of each of the ligand precursors that have been prepared is reported below. Ligand precursors [H₂L⁵]Br, [H₂L⁶]Br, [H₂L⁷]Br, [H₂L⁸]Br, [H₂L⁹]Br, [H₂L¹⁰]Br, [H₂L¹¹]Br, [H₂L¹²]Br, [H₂L¹³]Cl, [H₂L¹⁴]Br, [H₂L¹⁵]Br, [H₂L¹⁶]Br, [H₂L¹⁷]Br, [H₂L¹⁸]Br, [H₂L¹⁹]Cl, [H₂L²⁰]Cl, and [H₂L²¹]Cl are within the scope of the present application and ligand precursors [H₂L¹]Br, [H₂L²]Br, [H₂L³]Br, and [H₂L⁴]Br were prepared for comparison purposes.

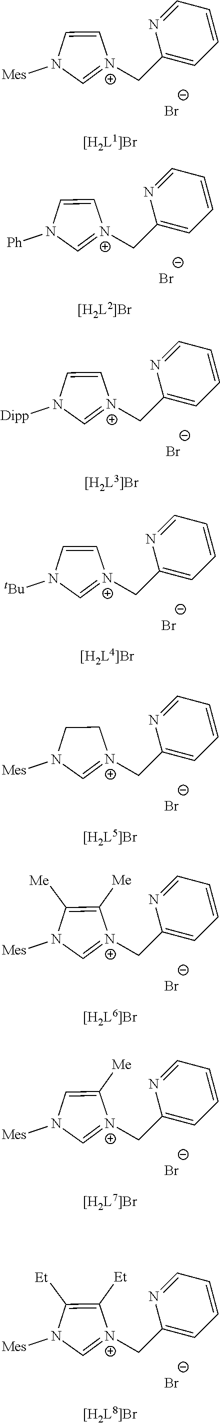

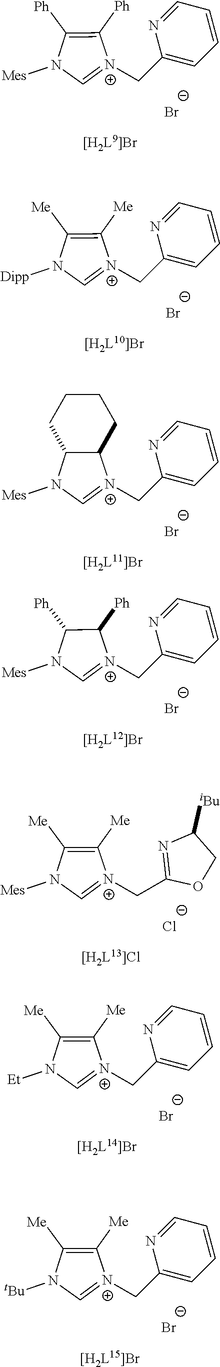

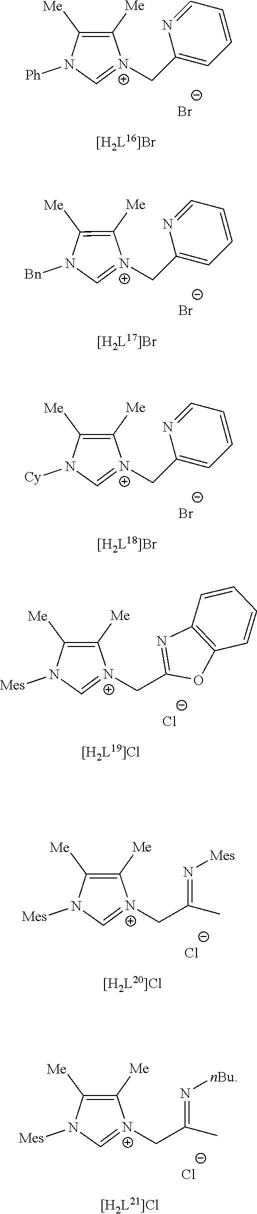

3-Mesityl-1-(2-pyridinylmethyl)-imidazolium bromide [H₂L¹]Br, 3-phenyl-1-(2-pyridinylmethyl)-imidazolium bromide [H₂L²]Br, 3-(2,6-diisopropylphenyl)-1-(2-bromide [H₂L³]Br and pyridinylmethyl)-imidazolium 3-(tert-butyl)-1-(2-pyridinylmethyl) imidazolium bromide [H₂L⁴]Br were synthesized according to the literature ([H₂L¹]Br, [H₂L³]Br and [H₂L⁴]Br: Tulloch A. A. D., Danopoulos A. A., Winston S., Kleinhenz S., Eastham G. J. Chem. Soc., Dalton Trans. 2000, 4499-4506; [H₂L²]Br: Fernández F. E., Puerta M. C., Valerga P. Organometallics, 2011, 30, 5793-5802).

Scheme 2 represents the general synthetic pathway for preparing the ligand precursors [H₂L⁵]Br, [H₂L⁶]Br, [H₂L⁷]Br, [H₂L⁸]Br, [H₂L⁹]Br, [H₂L¹⁰]Br, [H₂L¹¹]Br, [H₂L¹²]Br, [H₂L¹⁴]Br, [H₂L¹⁵]Br, [H₂L¹⁶]Br, [H₂L¹⁷]Br, and [H₂L¹⁸]Br.

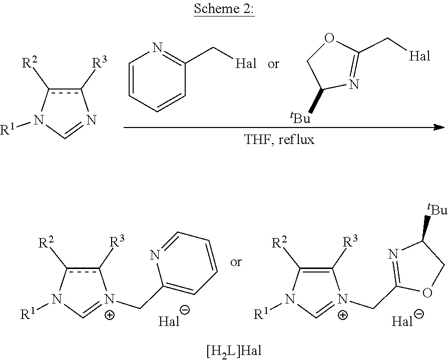

2-(Bromomethyl) pyridine hydrobromide (2.53 g, 10 mmol) was neutralized using a cold (0° C.) saturated aqueous solution of sodium carbonate. The liberated 2-bromomethylpyridine was extracted into cold (0° C.) diethyl ether (3×50 mL), dried with MgSO₄ and filtered. The filtrate was concentrated to ˜50 mL. The corresponding imidazole (10 mmol) in THF (50 mL) was added, the ether was removed under reduced pressure and the solution was refluxed overnight. All volatiles were removed under vacuum and 50 mL of ethyl acetated were added into the residue giving a suspension. The product was collected by filtration, washed with ethyl acetate (3×20 mL) and dried under vacuum.

[H₂L⁵]Br. By the general procedure using 1-mesityl-4,5-dihydroimidazole³ (1.88 g, 10 mmol). The collected solid contained ˜30% of 1-mesityl-4,5-dihydroimidazolium bromide. It was dissolved in acetonitrile and stirred with solid Na₂CO₃ overnight, filtered. The filtration was concentrated to dryness under reduced pressure to afford a white solid. The solid was washed with diethyl ether (3×20 mL) and dried under vacuum. Yield: 1.59 mg, 44%. ¹H NMR (500 MHZ, CDCl₃): δ 9.54 (s, 1H, dihydroimidazolium-H), 8.52 (ddd, J=4.9, 1.8, 1.0 Hz, 1H, py-H), 7.71 (td, J=7.7, 1.8 Hz, 1H, py-H), 7.59 (dt, J=7.9, 1.1 Hz, 1H, py-H), 7.25 (ddd, J=7.5, 4.8, 1.2 Hz, 1H, py-H), 6.95-6.86 (m, 2H, Mes-H), 5.35 (s, 2H, CH₂), 4.37-4.22 (m, 2H, dihydroimidazolium-CH₂), 4.23-4.07 (m, 2H, dihydroimidazolium-CH2), 2.33 (s, 6H, Mes-CH₃), 2.26 (s, 3H, Mes-CH₃). ¹³C NMR (126 MHZ, CDCl₃): δ 160.61 (dihydroimidazolium-C), 152.93 (py-C), 149.67 (py-C), 140.43 (Mes-C), 137.55 (py-C), 135.45 (Mes-C), 130.66 (Mes-C), 130.07 (Mes-C), 123.69 (py-C), 123.58 (py-C), 52.70 (CH₂), 51.19 (dihydroimidazolium-CH₂), 49.63 (dihydroimidazolium-CH₂), 21.11 (Mes-CH₃), 18.11 (Mes-CH₃).

[H₂L⁶]Br. By the general procedure using 1-mesityl-4,5-dimethyl-imidazole⁴ (2.14 g, 10 mmol). Yield: 3.38 g, 88%. ¹H NMR (600 MHZ, CDCl₃): δ 9.95 (s, 1H, imidazolium-H), 8.46 (ddd, J=4.9, 1.8, 0.9 Hz, 1H, py-H), 7.83 (d, J=7.8 Hz, 1H, py-H), 7.76 (td, J=7.7, 1.8 Hz, 1H, py-H), 7.27 (ddd, J=7.5, 4.9, 1.2 Hz, 1H, py-H), 6.99 (d, J=1.1 Hz, 2H, Mes-H), 6.08 (s, 2H, CH₂), 2.34 (d, J=0.9 Hz, 3H, imidazolium-CH₃), 2.33 (s, 3H, Mes-CH₃), 2.00 (s, 6H, Mes-CH₃), 1.93 (d, J=0.9 Hz, 3H, imidazolium-CH₃). ¹³C NMR (126 MHZ, CDCl₃): δ 152.94 (py-C), 149.18 (py-C), 141.33 (Mes-C), 138.13 (py-C), 136.83 (imidazolium-C), 135.04 (Mes-C), 129.95 (Mes-C), 129.05 (Mes-C), 128.14 (imidazolium-C), 126.81 (imidazolium-C), 123.85 (py-C), 123.80 (py-C), 51.52 (CH₂), 21.23 (Mes-CH₃), 17.69 (Mes-CH₃), 9.38 (imidazolium-CH₃), 8.42 (imidazolium-CH₃). HRMS (ESI): m/z calcd for C₂₀H₂₄N₃ [M-Br]⁺306.1965, found 306.1965.

[H₂L⁷]Br. By the general procedure using 4-methyl-1-mesityl-imidazole⁴ (2.00 g, 10 mmol). Yield: 2.85 g, 77%. ¹H NMR (500 MHZ, CDCl₃): δ 10.14 (d, J=1.7 Hz, 1H, imidazolium-H), 8.49 (dd, J=5.1, 1.7 Hz, 1H, py-H), 7.93 (d, J=7.8 Hz, 1H, py-H), 7.83 (td, J=7.7, 1.8 Hz, 1H, py-H), 7.32 (ddd, J=7.6, 4.9, 1.0 Hz, 1H, py-H), 6.99 (s, 2H, Mes-H), 6.86 (s, 1H, imidazolium-H), 6.13 (s, 2H, CH₂), 2.46 (d, J=1.1 Hz, 3H, imidazolium-CH₃), 2.34 (s, 3H, Mes-CH₃), 2.11 (s, 6H, CH₃). ¹³C NMR (126 MHZ, CDCl₃): δ 152.62 (py-C), 149.06 (py-C), 141.31 (Mes-C), 138.94 (py-C), 138.49 (imidazolium-C), 134.54 (Mes-C), 132.96 (imidazolium-C), 130.92 (Mes-C), 129.93 (Mes-C), 124.14 (py-C), 124.09 (py-C), 119.58 (imidazolium-C), 51.34 (CH₂), 21.24 (Mes-CH₃), 17.78 (Mes-CH₃), 10.06 (imidazolium-CH₃). HRMS (ESI): m/z calcd for C₁₉H₂₂N_{3 [}M-Br]⁺ 292.1808, found 292.1815.

[H₂L⁸]Br. By the general procedure using 4,5-diethyl-1-mesityl-imidazole⁵ (2.42 g, 10 mmol). Yield: 2.98 g, 72%. ¹H NMR (600 MHZ, CDCl₃): δ 10.01 (s, 1H, imidazolium-H), 8.45 (ddd, J=4.8, 1.8, 0.9 Hz, 1H, py-H), 7.79 (dt, J=7.8, 1.1 Hz, 1H, py-H), 7.73 (td, J=7.6, 1.8 Hz, 1H, py-H), 7.23 (ddd, J=7.5, 4.8, 1.2 Hz, 1H, py-H), 7.00 (q, J=0.7 Hz, 2H, Mes-H), 6.06 (s, 2H, CH₂), 2.80 (q, J=7.6 Hz, 2H, imidazolium-CH₂CH₃), 2.37 (q, J=7.6 Hz, 2H, imidazolium-CH₂CH₃), 2.34 (s, 3H, Mes-CH₃), 2.04 (s, 6H, Mes-CH₃), 1.14 (t, J=7.6 Hz, 3H, imidazolium-CH₂CH₃), 0.96 (t, J=7.6 Hz, 3H, imidazolium-CH₂CH₃). ¹³C NMR (126 MHZ, CDCl₃): δ 153.61 (py-C), 149.60 (py-C), 141.29 (Mes-C), 137.61 (py-C), 137.50 (imidazolium-C), 135.10 (Mes-C), 133.02 (imidazolium-C), 131.88 (imidazolium-C), 130.01 (Mes-C), 129.16 (Mes-C), 123.64 (py-C), 123.42 (py-C), 51.74 (CH₂), 21.26 (Mes-CH₃), 17.76 (Mes-CH₃), 16.75 (imidazolium-CH₂CH₃), 16.58 (imidazolium-CH₂CH₃), 14.09 (imidazolium-CH₂CH₃), 13.54 (imidazolium-CH₂CH₃). HRMS (ESI): m/z calcd for C₂₂H₂₈N₃ [M-Br]⁺334.2278, found 334.2277.

[H₂L⁹]Br. By the general procedure using 4,5-diphenyl-1-mesityl-imidazole⁵ (3.38 g, 10 mmol). Yield: 2.98 g, 72%. ¹H NMR (600 MHZ, CDCl₃): δ 10.07 (s, 1H, imidazolium-H), 8.47 (ddd, J=4.9, 1.8, 0.9 Hz, 1H, py-H), 7.66 (td, J=7.7, 1.8 Hz, 1H, py-H), 7.44-7.41 (m, 2H, overlapping, py-H and Ph-H), 7.37-7.30 (m, 4H, Ph-H), 7.26-7.20 (m, 2H, overlapping, py-H and Ph-H), 7.13 (dd, J=8.7, 7.1 Hz, 2H, Ph-H), 6.94-6.90 (m, 4H, overlapping, Ph-H and Mes-H), 6.08 (s, 2H, CH₂), 2.29 (s, 3H, Mes-CH₃), 2.16 (s, 6H, Mes-CH₃). ¹³C NMR (126 MHZ, CDCl₃): δ 153.24 (py-C), 149.36 (py-C), 141.16 (Mes-C), 139.19 (imidazolium-C), 137.51 (py-C), 135.23 (Mes-C), 132.78 (imidazolium-C), 131.46 (imidazolium-C), 131.15 (Ph-C), 130.63 (Ph-C), 129.92 (Ph-C), 129.90 (Ph-C), 129.83 (Mes-C), 129.66 (Mes-C), 129.45 (Ph-C), 129.30 (Ph-C), 128.83 (Ph-C), 125.30 (Ph-C), 125.16 (Ph-C), 123.53 (py-C), 123.15 (py-C), 51.83 (CH₂), 21.25 (Mes-CH₃), 18.33 (Mes-CH₃).

[H₂L¹⁰]Br. By the general procedure using 4,5-dimethyl-1-(2,6-diisopropylphenyl)-imidazole⁵ (2.56 g, 10 mmol). Yield: 3.54 g, 83%. ¹H NMR (600 MHZ, CDCl₃): δ 10.08 (s, 1H, imidazolium-H), 8.51-8.40 (m, 1H, py-H), 7.91 (d, J=7.8 Hz, 1H, py-H), 7.78 (td, J=7.7, 1.8 Hz, 1H, py-H), 7.53 (t, J=7.8 Hz, 1H, Dipp-H), 7.31 (d, J=7.8 Hz, 2H, Dipp-H), 7.28 (ddd, J=7.6, 4.9, 1.1 Hz, 1H, py-H), 6.19 (s, 2H, CH₂), 2.40 (s, 3H, imidazolium-CH₃), 2.27 (hept, J=6.8 Hz, 2H, CH (CH₃)₂), 1.95 (s, 3H, imidazolium-CH₃), 1.19 (d, J=6.8 Hz, 6H, CH (CH₃)₂), 1.17 (d, J=6.9 Hz, 6H, CH (CH₃)₂). ¹³C NMR (126 MHZ, CDCl₃): δ 153.07 (py-C), 149.01 (py-C), 146.02 (Dipp-C), 138.24 (py-C), 137.51 (imidazolium-C), 132.01, 128.48, 128.10, 127.54 (imidazolium-C), 124.97 (Dipp-C), 124.04 (py-C), 123.94 (py-C), 51.46 (CH₂), 28.80 (CH (CH₃)₂), 25.36 (CH (CH₃)₂), 23.34 (CH (CH₃)₂), 9.52 (imidazolium-CH₃), 8.78 (imidazolium-CH₃).

[H₂L¹¹]Br. By the general procedure using (3R,7R)-1-mesityl-hexahydrobenzoimidazole (1.21 g, 5 mmol) and 2-(bromomethyl) pyridine hydrobromide (1.27 g, 5 mmol). Yield: 1.55 g, 75%.

[H₂L¹²]Br. By the general procedure using (4R,5R)-1-mesityl-4,5-diphenyl-4,5-dihydroimidazole⁶ (1.70 g, 5 mmol) and 2-(bromomethyl) pyridine hydrobromide (1.27 g, 5 mmol). Yield: 2.01 g, 78%. ¹H NMR (500 MHZ, CDCl₃) δ 10.01 (s, 1H, dihydroimidazolium-H), 8.69 (ddd, J=4.9, 1.7, 0.8 Hz, 1H, py-H), 7.75 (td, J=7.7, 1.8 Hz, 1H, py-H), 7.52-7.40 (m, 6H, overlapping, 5 Ph-H and 1 py-H), 7.39-7.28 (m, 6H, 5 Ph-H and 1 py-H), 6.87 (s, 1H, Mes-H), 6.68 (s, 1H, Mes-H), 6.31 (d, J=16.3 Hz, 1H, CH₂), 5.97-5.72 (m, 1H, dihydroimidazolium-H), 5.20 (d, J=8.7 Hz, 1H, dihydroimidazolium-H), 4.59 (d, J=16.2 Hz, 1H, CH₂), 2.47 (s, 3H, Mes-CH₃), 2.19 (s, 3H, Mes-CH₃), 1.86 (s, 3H, Mes-CH₃). ¹³C NMR (126 MHZ, CDCl₃) δ 160.26 (dihydroimidazolium-C), 153.44 (py-C), 149.19 (py-C), 140.10 (Mes-C), 137.84 (py-C), 136.36 (Mes-C), 135.38 (Ph-C), 134.99 (Mes-C), 133.84 (Ph-C), 130.42 (Ph-C), 130.34 (Ph-C), 130.12 (Mes-C), 130.09 (Mes-C), 129.24 (Ph-C), 129.06 (iMes-C), 127.34 (Ph-C), 123.67 (py-C), 123.43 (py-C), 75.71 (dihydroimidazolium-CH-Ph), 71.49 (dihydroimidazolium-CH-Ph), 50.60 (CH₂), 21.02 (Mes-CH₃), 19.13 (Mes-CH3), 18.14 (Mes-CH₃).

[H₂L¹⁴]Br. By the general procedure using 4,5-dimethyl-1-ethyl-imidazole (1.24 g, 10 mmol). Yield: 2.54 g, 86%. ¹H NMR (600 MHZ, CDCl₃): δ 10.48 (s, 1H, imidazolium-H), 8.51 (ddd, J=5.0, 1.8, 0.9 Hz, 1H,, py-H), 7.92 (d, J=7.8 Hz, 1H,, py-H), 7.85 (td, J=7.7, 1.8 Hz, 1H, py-H), 7.37-7.32 (m, 1H, py-H), 5.80 (s, 2H, CH₂), 4.19 (q, J=7.3 Hz, 2H, CH₂CH₃), 2.27 (d, J=0.8 Hz, 3H, imidazolium-CH₃), 2.25 (d, J=0.9 Hz, 3H, imidazolium-CH₃), 1.59 (t, J=7.3 Hz, 3H, CH₂CH₃).). ¹³C NMR (126 MHZ, CDCl₃): δ 152.58 (py-C), 148.71 (py-C), 138.99 (py-C), 136.45 (imidazolium-C), 127.76 (imidazolium-C), 126.05 (imidazolium-C), 124.47 (py-C), 124.23 (py-C), 51.21 (CH₂), 42.83 (CH₂CH₃), 15.23 (CH₂CH₃), 9.09 (imidazolium-CH₃), 8.64 (imidazolium-CH₃).

[H₂L¹⁵]Br. By the general procedure using 4,5-dimethyl-1-tert-butyl-imidazole (1.52 g, 10 mmol). Yield: 2.17 g, 67%. ¹H NMR (500 MHZ, CDCl₃): δ 10.39 (s, 1H, imidazolium-H), 8.45 (ddd, J=4.8, 1.8, 0.9 Hz, 1H, py-H), 7.86 (dt, J=7.8, 1.1 Hz, 1H, py-H), 7.72 (td, J=7.7, 1.8 Hz, 1H, py-H), 7.22 (ddd, J=7.6, 4.9, 1.2 Hz, 1H, py-H), 5.83 (s, 2H, CH₂), 2.39 (s, 3H, imidazolium-CH₃), 2.25 (s, 3H, imidazolium-CH₃), 1.74 (s, 9H, tert-butyl-CH₃). ¹³C NMR (126 MHz, CDCl₃): δ 153.50 (py-C), 149.41 (py-C), 137.73 (py-C), 135.71 (imidazolium-C), 129.43 (imidazolium-C), 125.71 (imidazolium-C), 124.03 (py-C), 123.66 (py-C), 61.38 (^{t}Bu-C), 51.60 (CH₂), 30.00 (^{t}Bu-CH₃), 11.90 (imidazolium-CH₃), 9.05 (imidazolium-CH₃).

[H₂L¹⁶]Br. By the general procedure using 4,5-dimethyl-1-phenyl-imidazole (1.72 g, 10 mmol). Yield: 2.44 g, 70%. ¹H NMR (600 MHZ, CDCl₃): δ 10.21 (s, 1H, imidazolium-H), 8.47 (ddd, J=4.8, 1.8, 0.9 Hz, 1H, py-H), 7.84 (dt, J=7.7, 1.1 Hz, 1H, py-H), 7.72 (td, J=7.7, 1.8 Hz, 1H, py-H), 7.55-7.51 (m, 3H, Ph-H), 7.50-7.46 (m, 2H, Ph-H), 7.23 (ddd, J=7.6, 4.9, 1.1 Hz, 1H, py-H), 5.95 (s, 2H, CH₂), 2.34 (d, J=0.9 Hz, 3H, imidazolium-CH₃), 2.15 (d, J=0.9 Hz, 3H, imidazolium-CH₃). ¹³C NMR (151 MHZ, CDCl₃): δ 153.09 (py-C), 149.65 (py-C), 137.68 (py-C), 136.36 (imidazolium-C), 133.34 (Ph-C), 130.79 (Ph-C), 130.37 (Ph-C), 128.20 (imidazolium-C), 126.64 (imidazolium-C), 125.92 (Ph-C), 123.91 (py-C), 123.75 (py-C), 51.90 (CH₂), 9.43 (imidazolium-CH₃), 9.27 (imidazolium-CH₃).

[H₂L¹⁷]Br. By the general procedure using 4,5-dimethyl-1-benzyl-imidazole (1.86 g, 10 mmol). Yield: 2.86 g, 80%. ¹H NMR (500 MHZ, CDCl³): δ 10.60 (s, 1H, imidazolium-H), 8.46 (ddd, J=4.8, 1.8, 1.0 Hz, 1H, py-H), 7.72 (td, J=7.6, 1.8 Hz, 1H, py-H), 7.67 (dt, J=7.8, 1.2 Hz, 1H, py-H), 7.36-7.27 (m, 5H, Ph-H), 7.23 (ddd, J=7.4, 4.8, 1.3 Hz, 1H, py-H), 5.68 (s, 2H, CH₂), 5.45 (s, 2H, CH₂-Ph), 2.18 (d, J=0.9 Hz, 3H, imidazolium-CH₃), 2.09 (d, J=0.9 Hz, 3H, imidazolium-CH₃). ¹³C NMR (126 MHZ, CDCl₃): δ 152.85 (py-C), 149.72 (py-C), 137.73 (py-C), 137.05 (Ph-C), 133.01 (Ph-C), 129.42 (Ph-C), 129.04 (imidazolium-C), 128.01 (imidazolium-C), 127.90 (Ph-C), 126.67 (imidazolium-C), 123.78 (py-C), 123.30 (py-C), 51.89 (CH₂), 51.21 (CH₂-Ph), 8.99 (imidazolium-CH₃), 8.97 (imidazolium-CH₃).

[H₂L¹⁸]Br. By the general procedure using 4,5-dimethyl-1-cyclohexyl-imidazole (1.92 g, 10 mmol). Yield: 3.21 g, 88%. ¹H NMR (600 MHZ, CDCl₃): δ 10.55 (s, 1H, imidazolium-H), 8.47 (ddd, J=4.8, 1.8, 0.9 Hz, 1H, py-H), 7.92 (d, J=7.8 Hz, 1H, py-H), 7.77 (td, J=7.7, 1.8 Hz, 1H, py-H), 7.27 (ddd, J=7.7, 4.9, 1.2 Hz, 1H, py-H), 5.83 (s, 2H, CH₂), 3.91 (tt, J=11.9, 3.6 Hz, 1H, Cy-CH), 2.28 (d, J=0.9 Hz, 3H, imidazolium-CH₃), 2.25 (d, J=0.9 Hz, 3H, imidazolium-CH₃), 2.10 (dd, J=11.8, 3.1 Hz, 2H, Cy-CH₂), 1.95 (ddp, J=15.5, 12.3, 3.8 Hz, 4H, Cy-CH₂), 1.72 (d, J=8.5 Hz, 1H, Cy-CH₂), 1.38 (td, J=11.0, 2.8 Hz, 3H, Cy-CH₂). ¹³C NMR (126 MHZ, CDCl₃): δ 153.10 (py-C), 148.82 (py-C), 138.58 (py-C), 135.34 (imidazolium-C), 127.57 (imidazolium-C), 125.43 (imidazolium-C), 124.57 (py-C), 124.01 (py-C), 58.19 (Cy-CH), 51.31 (CH₂), 33.42 (Cy-CH₂), 25.58 (Cy-CH₂), 24.57 (Cy-CH₂), 9.07 (imidazolium-CH₃), 8.89 (imidazolium-CH₃).

Scheme 3 represents the synthetic pathway for preparing the ligand precursor [H₂L¹³]Cl

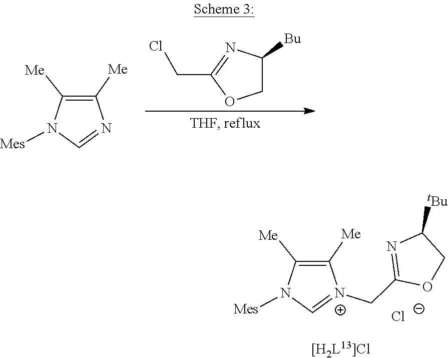

[H₂L¹³]Cl. 1-mesityl-4,5-dihydroimidazole (1.88 g, 10 mmol; prepared according to Paczal A. et al. J. Org. Chem. 2006, 71, 5969-5979) and(S)-2-(chloromethyl)-4-isobutyl-4,5-dihydrooxazole (1.76 g, 10 mmol; prepared according to Kamata K. et al. Heterocycles 1999, 51, 373-378) were dissolved in THF (50 mL). The solution was refluxed overnight. All volatiles were removed under vacuum and 50 mL of ethyl acetate were added into the residue giving a suspension. The product was collected by filtration, washed with ethyl acetate (3×20 mL) and dried under vacuum. Yield: 2.95 g, 76%. ¹H NMR (500 MHZ, CDCl₃) δ 10.31 (s, 1H, imidazolium-H), 7.00 (s, 1H, Mes-H), 5.75 (dd, J=17.3, 1.3 Hz, 1H, CH₂), 5.68 (dd, J=17.2, 1.8 Hz, 1H, CH₂), 4.42 (dd, J=9.3, 8.2 Hz, 1H, dihydrooxazole-CH₂), 4.10 (dtd, J=9.2, 7.4, 5.8 Hz, 1H, dihydrooxazole-CH), 3.90 (t, J=8.1 Hz, 1H, dihydrooxazole-CH₂), 2.34 (s, 3H, Mes-CH₃), 2.29 (d, J=0.9 Hz, 3H, imidazolium-CH₃), 2.00 (s, 3H, Mes-CH₃), 1.99 (s, 3H, Mes-CH₃), 1.94 (d, J=0.9 Hz, 3H, imidazolium-CH₃), 1.66 (dt, J=13.4, 6.7 Hz, 1H, isobutyl-CH), 1.49 (dt, J=13.4, 7.1 Hz, 1H, isobutyl-CH₂), 1.27 (dt, J=13.5, 7.2 Hz, 1H, isobutyl-CH₂), 0.90 (d, J=5.9 Hz, 3H, isobutyl-CH₃), 0.88 (d, J=6.0 Hz, 3H, isobutyl-CH₃).

Schema 4 represents the synthetic pathway for preparing the ligand precursor [H₂L¹⁹]Cl

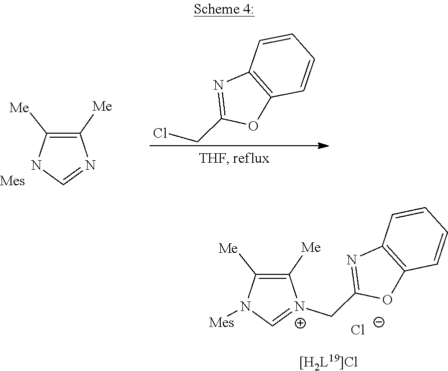

[H₂L¹⁹]Cl. 1-mesityl-4,5-dimethyl-imidazole (1.07 g, 5 mmol) and 2-(Chloromethyl)-benzo [d] oxazole (0.835 g, 5 mmol) were dissolved in THF (50 mL). The solution was refluxed overnight. All volatiles were removed under vacuum and 50 mL of ethyl acetate were added into the residue giving a suspension. The product was collected by filtration, washed with ethyl acetate (3×20 mL) and dried under vacuum. Yield: 1.53 g, 80%. ¹H NMR (600 MHZ, CDCl₃): δ 10.50 (s, 1H, imidazolium-H), 7.66-7.63 (m, 1H, benzoxazole-CH), 7.52-7.50 (m, 1H, benzoxazole-CH), 7.38-7.32 (m, 2H, benzoxazole-CH), 7.02 (s, 2H, Mes-CH), 6.56 (s, 2H, CH₂), 2.35 (s, 3H, imidazolium-CH₃), 2.34 (s, 3H, imidazolium-CH₃), 2.06 (s, 6H, Mes-CH₃), 1.98 (s, 3H, Mes-CH₃).). ¹³C NMR (126 MHZ, CDCl₃): δ 151.39 (benzoxazole-C), 148.78 (benzoxazole-C), 141.52 (benzoxazole-C), 137.99 (imidazolium-C), 135.04 (Mes-C), 130.03 (Mes-C), 129.15 (Mes-C), 129.15 (Mes-C), 127.86 (imidazolium-C), 127.30 (imidazolium-C), 125.97 (benzoxazole-CH), 124.94 (benzoxazole-CH), 120.40 (benzoxazole-CH), 111.24 (benzoxazole-CH), 44.83 (CH₂), 21.27 (Mes-CH₃), 17.75 (Mes-CH₃), 9.04 (imidazolium-CH₃), 9.48 (imidazolium-CH₃).

Schema 5 represents the general synthetic pathway for preparing the ligand precursors [H₂L²⁰]Cl and [H²L²¹]Cl

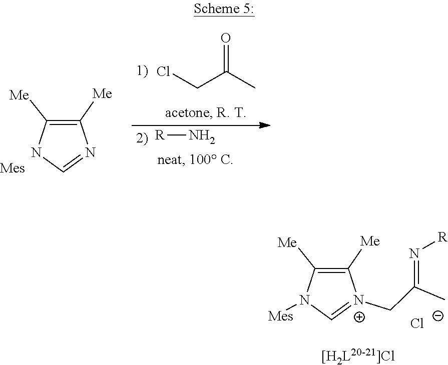

[H₂L²⁰]Cl. Chloroacetone (0.40 mL, 5 mmol) was added to a solution of 1-mesityl-4,5-dimethyl-imidazole (1.07 g, 5 mmol) in acetone (50 mL) and the mixture was stirred at room temperature for 2 hours. All volatiles were removed under vacuum and 2,4,6-trimethylaniline (3.5 mL, 25 mmol) was added to the reaction flask. The mixture was stirred at 100° C. for 36 hours. The product was purified by column chromatography on basic alumina (DCM: MeOH 100:0 v/v, increasing to 90:10 v/v) to give the pure product. Yield: 1.4 g, 66%. ¹H NMR (600 MHZ, CDCl₃): δ 10.11 (s, 1H, imidazolium-H), 6.98 (s, 2H, Mes-CH), 6.79 (s, 2H, Mes-CH), 5.89 (s, 2H, CH₂), 2.36 (d, J=0.9 Hz, 3H, imidazolium-CH₃), 2.33 (s, 3H, Mes-CH₃), 2.22 (s, 3H, Mes-CH₃), 1.97 (s, 6H, Mes-CH₃), 1.93 (d, J=0.9 Hz, 3H, imidazolium-CH₃), 1.89 (s, 6H, Mes-CH₃), 1.88 (s, 4H, CH₃). ¹³C NMR (126 MHZ, CDCl₃): δ 164.90 (CN), 144.74 (Mes-C), 141.27 (Mes-C), 137.44 (imidazolium-C), 135.10 (Mes-C), 132.70 (Mes-C), 129.89 (Mes-C), 129.14 (Mes-C), 128.79 (Mes-C), 127.90 (imidazolium-C), 126.17 (imidazolium-C), 125.51 (Mes-C), 52.92 (CH₂), 21.25 (Mes-CH₃), 20.74 (Mes-CH₃), 18.60 (CH₃), 18.21 (Mes-CH₃), 14.33 (Mes-CH₃), 8.97 (imidazolium-CH₃), 8.41 (imidazolium-CH₃).

[H₂L²¹]Cl. Chloroacetone (0.40 mL, 5 mmol) was added to a solution of 1-mesityl-4,5-dimethyl-imidazole (1.07 g, 5 mmol) in acetone (50 mL) and the mixture was stirred at room temperature for 2 hours. All volatiles were removed under vacuum and n-butylamine (2.4 mL, 25 mmol) was added to the reaction flask. The mixture was stirred at 25° C. for 6 hours. The product was purified by column chromatography on basic alumina (DCM: MeOH 100:0 v/v, increasing to 90:10 v/v) to give the pure product. Yield: 1.5 g, 83%. ¹H NMR (600 MHz, CDCl₃): δ 9.93 (s, 1H, imidazolium-H), 6.99 (s, 2H, Mes-CH), 5.54 (s, 2H, CH₂), 3.23-3.18 (m, 2H, nBu-CH₂), 2.34 (s, 3H, Mes-CH₃), 2.17 (d, J=0.8 Hz, 3H, imidazolium-CH₃), 2.00 (s, 6H, Mes-CH₃), 1.99 (s, 3H, CH₃), 1.93 (d, J=0.9 Hz, 3H, imidazolium-CH₃), 1.62 (s, 5H), 1.45-1.38 (m, 2H, nBu-CH₂), 1.29-1.19 (m, 2H, nBu-CH₂), 0.86 (t, J=7.4 Hz, 3H, nBu-CH₃).

### Syntheses of Iron Complexes

The following iron-based complexes [1] to were synthesized. Iron-based complexes [5] to are within the scope of the present application and complexes [1] to [4] were prepared for comparison purposes.

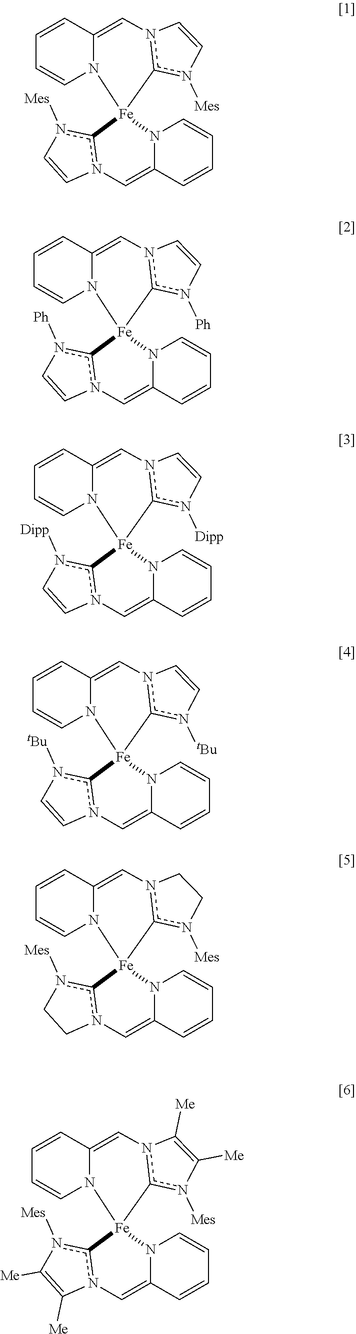

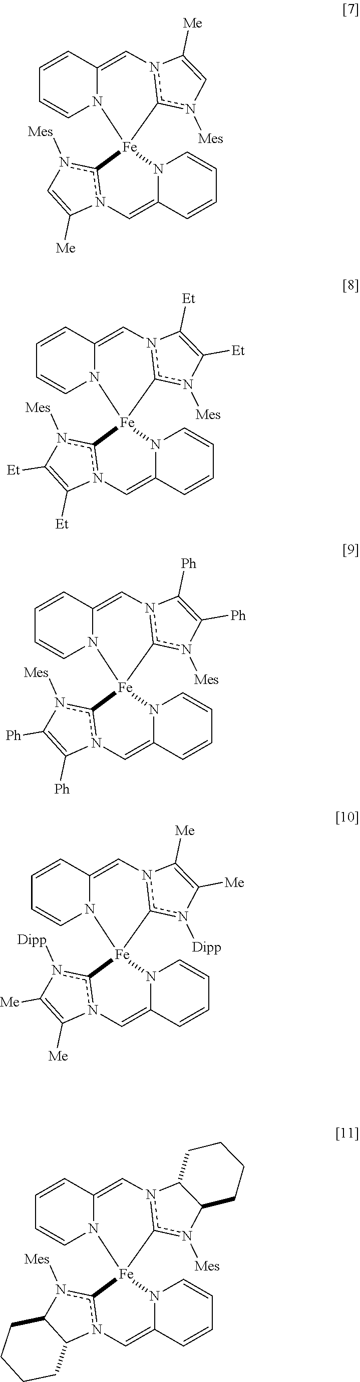

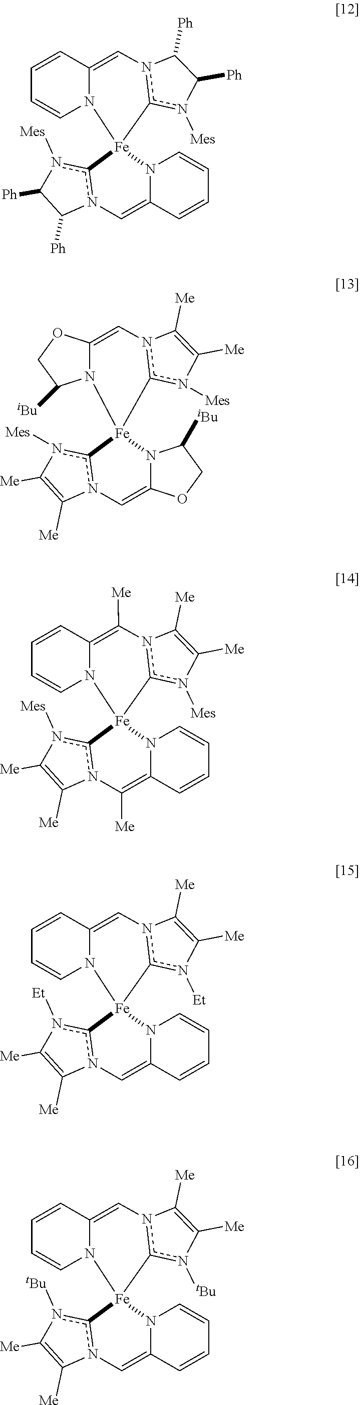

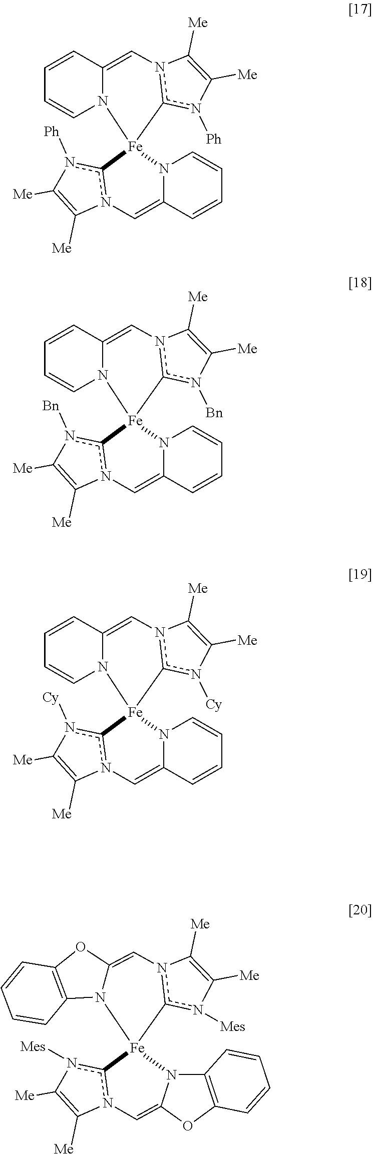

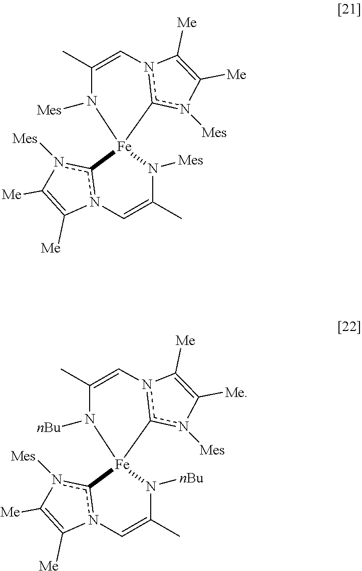

Complex [1] was synthesized according to the literature (Liang Q., Janes T., Gjergli X., Song D. Dalton Trans. 2016, 45, 13872-13880; Liang, Q.; Liu, N. J.; Song, D. Dalton Trans. 2018, 47, 9889-9696).

Scheme 6 below represents the general synthetic pathway for the preparation of iron-based complexes [2] to and to [22].

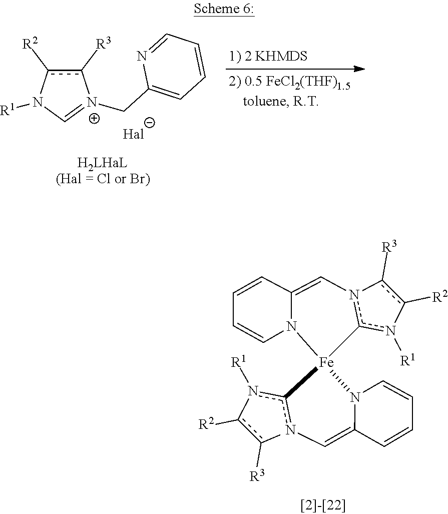

General procedure: To a stirring suspension of H₂LBr (0.50 mmol) and FeCl₂(THF)_{1.5} (58.5 mg, 0.25 mmol) in 2 mL of toluene was slowly added KHMDS (200 mg, 1.00 mmol) in 2 mL of toluene at room temperature. The reaction mixture was stirred overnight, and then concentrated to dryness under vacuum. The residue was dissolved in 3 mL of toluene and filtered through Celite. The filtrate was concentrated to dryness under vacuum to afford a brown solid, which was washed with cold pentane (3×1 mL) and dried under vacuum.

Complex [2]. By the general procedure using [H₂L²]Br (158.1 mg, 0.5 mmol), [2] was obtained as a brown solid (98.6 mg, 75%). ¹H NMR (600 MHZ, C₆D₆): δ 84.50 (2H), 64.65 (2H), 30.34 (2H), 17.87 (4H), 15.95 (2H), 14.38 (2H), −0.40 (4H), −56.58 (2H), −104.00 (2H).

Complex [3]. By the general procedure using [H₂L³]Br (200.2 mg, 0.5 mmol), [3] was obtained as a brown solid (113.2 mg, 65%). ¹H NMR (600 MHZ, C₆D₆): δ 81.79 (2H), 63.27 (2H), 22.44 (2H), 17.97 (2H), 6.00 (2H), 3.38 (2H), 0.78 (6H), 0.63 (2H)), −5.41 (6H), −5.86 (6H), −6.60 (6H), −33.91 (2H), −55.83 (2H), −129.31 (2H).

Complex [4]. By the general procedure using [H₂L⁴]Br (148.1 mg, 0.5 mmol), [4] was obtained as brown crystals (109.2 mg, 90%). ¹H NMR (600 MHZ, C₆D₆): δ 82.56 (2H), 58.24 (2H), 35.68 (2H), 15.90 (2H), 5.29 (18H), −46.21 (2H), −82.65 (2H).

Complex [5]. By the general procedure using [H₂L⁵]Br (180.2 mg, 0.5 mmol), [5] was obtained as brown crystals (118.9 mg, 78%). ¹H NMR (600 MHZ, C₆D₆): δ 103.88 (2H), 78.93 (2H), 65.44 (2H), 6.96 (2H), 1.64 (6H), 1.47 (2H), −0.98 (2H), −5.08 (6H), −8.47 (6H), −39.42 (2H), −82.10 (2H), −82.51 (2H), −168.35 (2H).

Complex [6]. By the general procedure using [H₂L⁶]Br (193.2 mg, 0.5 mmol), [6] was obtained as a brown solid (133.7 mg, 80%). ¹H NMR (600 MHZ, C₆D₆): δ 66.68 (2H), 64.55 (2H), 21.41 (2H), 3.80 (2H), 1.41 (6H), −0.20 (2H), −2.59 (6H), −7.71 (6H), −16.82 (6H), −55.43 (2H), −114.57 (2H).

Complex [7]. By the general procedure using [H₂L⁷]Br (186.2 mg, 0.50 mmol), [7] was obtained as a brown solid (119.1 mg, 75%). ¹H NMR (600 MHZ, C₆D₆): δ 62.79 (2H), 23.18 (2H), 16.23 (2H), 3.55 (2H), 1.26 (6H), 0.06 (2H), −2.30 (6H), −8.24 (6H), −15.10 (6H), −53.49 (2H), −114.36 (2H).

Complex [8]. By the general procedure using [H₂L⁸]Br (207.2 mg, 0.5 mmol), [8] was obtained as a brown solid (137.4 mg, 76%). ¹H NMR (600 MHZ, C₆D₆): δ 65.63 (2H), 46.61 (2H), 44.99 (2H), 19.64 (2H), 13.06 (2H), 7.99 (2H), 6.97 (6H), 4.59 (2H), 1.59 (6H), −0.26 (2H), −1.53 (6H), −6.20 (6H), −18.02 (6H), −56.26 (2H), −117.52 (2H).

Complex [9]. By the general procedure using [H₂L⁹]Br (255.2 mg, 0.5 mmol), [9] was obtained as a red brown solid (188.2 mg, 82%). ¹H NMR (600 MHZ, C₆D₆): δ 66.48 (2H), 19.20 (2H), 15.30 (4H), 10.66 (4H), 9.56 (2H), 9.41 (2H), 5.04 (2H), 1.76 (6H), −0.35 (2H), −1.38 (2H), −2.25 (4H), −5.83 (6H), −16.23 (6H), −57.47 (2H), −129.40 (2H).

Complex [10]. By the general procedure using [H₂L¹⁰]Br (214.2 mg, 0.5 mmol), was obtained as a brown solid (126.4 mg, 68%). ¹H NMR (600 MHZ, C₆D₆): δ 67.41 (6H), 62.70 (2H), 15.03 (2H), 4.85 (2H), 2.68 (2H), 1.36 (2H), 0.98 (2H), 0.25 (3H), −0.21 (3H), −0.97 (6H), −6.59 (9H, overlapping), −7.55 (6H), −34.54 (1H), −57.26 (2H), −121.18 (1H).

Complex [11]. By the general procedure using [H₂L¹¹]Br (207.2 mg, 0.5 mmol). After the reaction and solvent removal, the residue was extracted into pentane and filtered through Celite. The filtrate was concentrated to ˜1 mL and cooled to −35° C. to yield orange crystals of complex [11], which was washed with cold pentane (1 mL) and dried under high vacuum (116.7 mg, 65%).

Complex [12]. By the general procedure using [H2L12]Br (256.3 mg, 0.5 mmol). After the reaction and solvent removal, the residue was extracted into pentane and filtered through Celite. The filtrate was concentrated to ˜1 mL and cooled to −35° C. to yield orange crystals of complex [12], which was washed with cold pentane (1 mL) and dried under high vacuum (154.3 mg, 67%). 1H NMR (600 MHZ, C6D6): δ 114.78 (2H), 79.04 (4H), 76.47 (1H), 73.57 (1H), 15.67 (6H), 11.67 (2H), 9.96 (8H), −5.82 (8H), −7.96 (4H), −8.99 (8H), −11.31 (2H), −46.46 (1H), −83.29 (4H), −87.74 (2H), −178.31 (1H).

Complex [13]. By the general procedure using [H₂L¹³]Br (195.0 mg, 0.5 mmol). After the reaction and solvent removal, the residue was extracted into pentane and filtered through Celite. The filtrate was concentrated to ˜1 mL and cooled to −35° C. to yield orange crystals of complex [13], which was washed with cold pentane (1 mL) and dried under high vacuum (116.9 mg, 61%). ¹H NMR (600 MHZ, C₆D₆): δ 42.32 (3H), 40.66 (1H), 39.51 (3H), 22.44 (1H), 11.69 (1H), 5.95 (1H), 5.03 (1H), 2.37 (3H), 1.80 (3H), −0.96 (3H), −1.59 (2H), −1.88 (3H), −2.43 (3H), −3.46 (3H), −3.86 (3H), −4.37 (3H), −5.00 (3H), −13.83 (1H), −15.64 (2H), −17.16 (3H), −23.34 (1H), −31.29 (1H), −64.15 (1H), −86.72 (1H), −88.83 (1H).

Complex [15]. By the general procedure using [H₂L¹⁴]Br (148.1 mg, 0.5 mmol), was obtained as dark red crystals (86 mg, 80%). ¹H NMR (600 MHZ, C₆D₆): δ 67.86 (s, 6H), 65.00 (s, 2H), 31.83 (s, 2H), −2.00 (s, 6H), −13.86 (s, 4H), −56.08 (s, 2H), −88.29 (s, 2H).

Complex [16]. By the general procedure using [H₂L¹⁵]Br (161.6 mg, 0.5 mmol), was obtained as dark red crystals (121 mg, 90%). ¹H NMR (600 MHZ, C₆D₆): δ 61.77 (s, 6H), 48.72 (s, 2H), 41.85 (s, 18H), 0.78 (s, 6H), −29.79 (s, 2H), −68.50 (s, 2H).

Complex [17]. By the general procedure using [H₂L¹⁶]Br (172.1 mg, 0.5 mmol), was obtained as dark orange crystals (108 mg, 75%). ¹H NMR (600 MHZ, C₆D₆): δ 67.46 (s, 6H), 63.58 (s, 2H), 32.58 (s, 2H), 10.29 (s, 2H), 0.39 (s, 6H), −2.86 (s, 4H), −55.77 (s, 2H), −90.03 (s, 2H).

Complex [18]. By the general procedure using [H₂L¹⁷]Br (179.1 mg, 0.5 mmol), was obtained as brown crystals (100 mg, 66%). ¹H NMR (600 MHZ, C₆D₆): δ 69.16 (s, 6H), 65.98 (s, 2H), 30.38 (s, 2H), 3.53 (s, 2H), 2.28 (s, 4H), −2.46 (s, 6H), −6.62 (s, 2H), −58.82 (s, 2H), −93.09 (s, 2H).

Complex [19]. By the general procedure using [H₂L₁₈]Br (175.2 mg, 0.5 mmol), was obtained as red crystals (115 mg, 78%). ¹H NMR (600 MHZ, C₆D₆): δ 62.98 (s, 6H), 61.02 (s, 2H), 32.57 (s, 2H), 11.07 (s, 2H), 2.49 (s, 2H), 1.95 (s, 2H), 0.18 (s, 6H), −1.48 (s, 2H), −4.51 (s, 2H), −22.88 (s, 2H), −30.72 (s, 2H), −50.02 (s, 2H), −80.86 (s, 2H).

Complex [20]. By the general procedure using [H₂L^{19]}Cl (191.0 mg, 0.5 mmol), was obtained as dark red crystals (130 mg, 70%). ¹H NMR (600 MHZ, C₆D₆): δ 47.51 (s, 2H), 46.94 (s, 6H), 15.44 (s, 2H), 6.37 (s, 2H), 2.89 (s, 6H), 0.90 (s, 6H), −2.91 (s, 4H), −20.71 (s, 2H), −42.40 (s, 2H), −64.12 (s, 2H).

Complex [21]. By the general procedure using [H₂L²⁰]Cl (212.0 mg, 0.5 mmol), was obtained as orange crystals (128 mg, 62%). ¹H NMR (600 MHZ, C₆D₆): δ 47.81 (s, 6H), 37.99 (s, 2H), 35.60 (s, 6H), 21.64 (s, 2H), 3.37 (s, 2H), 0.39 (s, 6H), 0.13 (s, 2H), −0.43 (s, 4H), −1.64 (s, 2H), −11.77 (s, 0.73), −38.46 (s, 2H).

Complex [22]. By the general procedure using [H₂L²¹]Cl (181.0 mg, 0.5 mmol), was obtained as dark red crystals (144 mg, 82%). ¹H NMR (600 MHZ, C₆D₆): δ 48.82 (s, 6H), 5.67 (s, 2H), 4.04 (s, 2H), 2.07 (s, 2H), 1.63 (s, 6H), −0.74 (s, 6H), −1.86 (s, 2H), −2.33 (s, 2H), −5.30 (s, 6H), −13.94 (s, 2H), −50.54 (s, 2H).

Complex was prepared according to the synthetic pathway shown in Scheme 7.

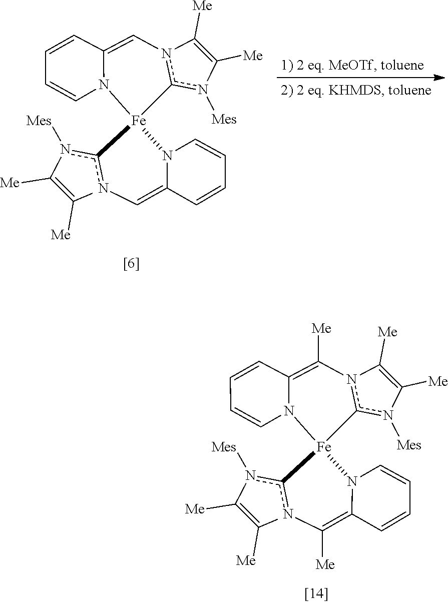

To a stirring suspension of [6] (166.2 mg, 0.25 mmol) in 5 mL of toluene was added MeOTf (60 μL, 0.55 mmol) at room temperature. The reaction mixture was stirred for an hour, affording a white suspension. All volatiles were removed under vacuum. To the solid residue was added 5 mL of toluene and then KHMDS (99.7 mg, 0.5 mmol) in 5 mL of toluene. The mixture was stirred overnight and then concentrated to dryness under vacuum. The residue was dissolved in 3 mL of toluene and filtered through Celite. The filtrate was concentrated to dryness under vacuum to afford a brown solid, which was washed with cold pentane (3×1 mL) and dried under vacuum. Yield: 125.7 mg, 73%. ¹H NMR (600 MHZ, C₆D₆) δ 174.18 (s, 4H), 60.06 (s, 6H), 56.37 (s, 2H), 15.64 (s, 2H), 4.40 (s, 6H), 3.47 (s, 2H), 1.89 (s, 6H), −0.46 (s, 2H), −9.86 (s, 6H), −15.23 (s, 6H), −46.66 (s, 2H).

### X-Ray Diffraction

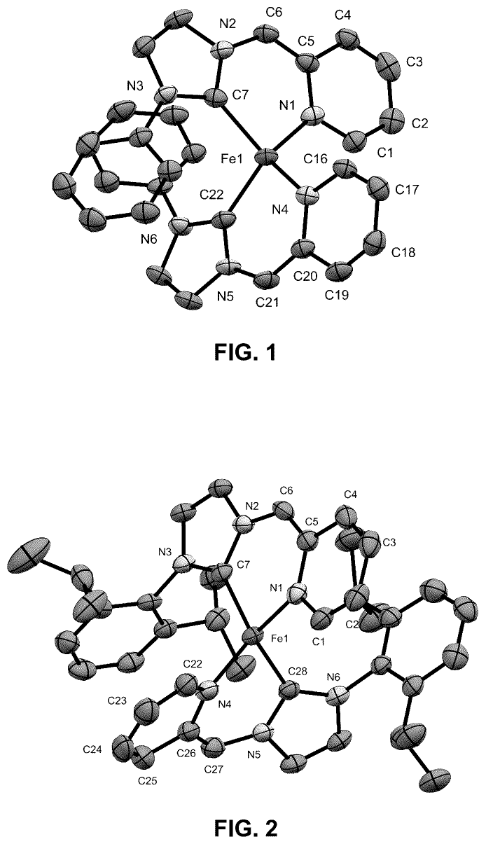

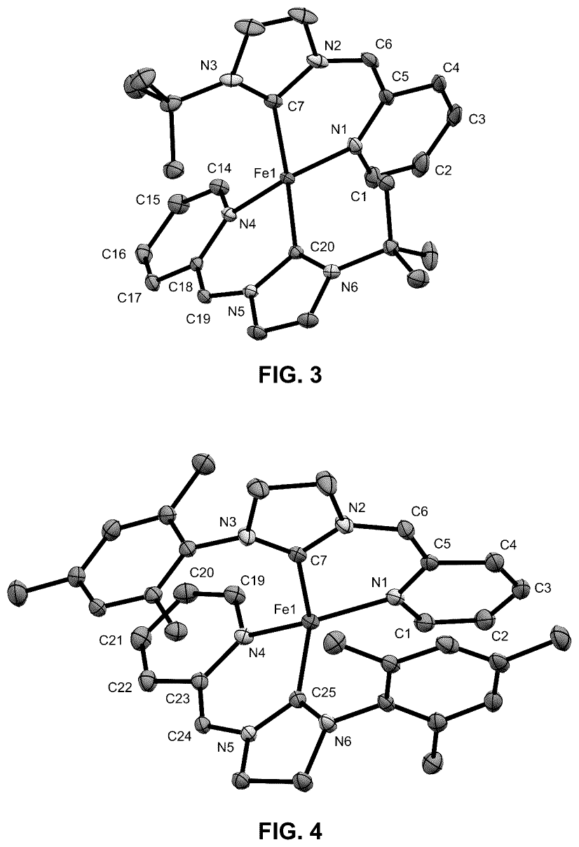

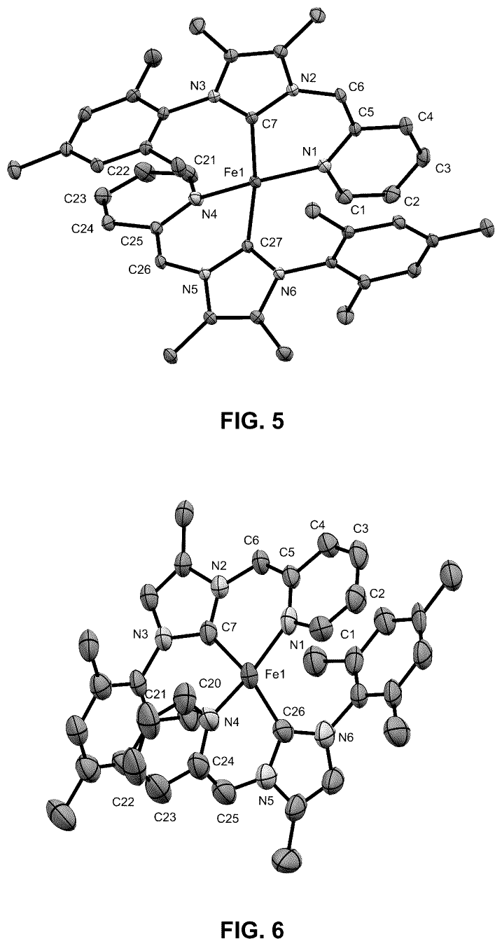

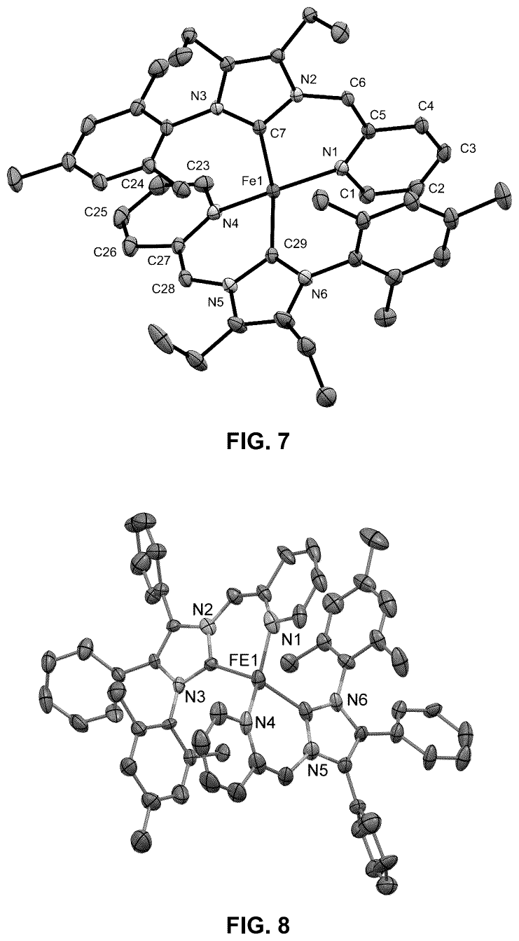

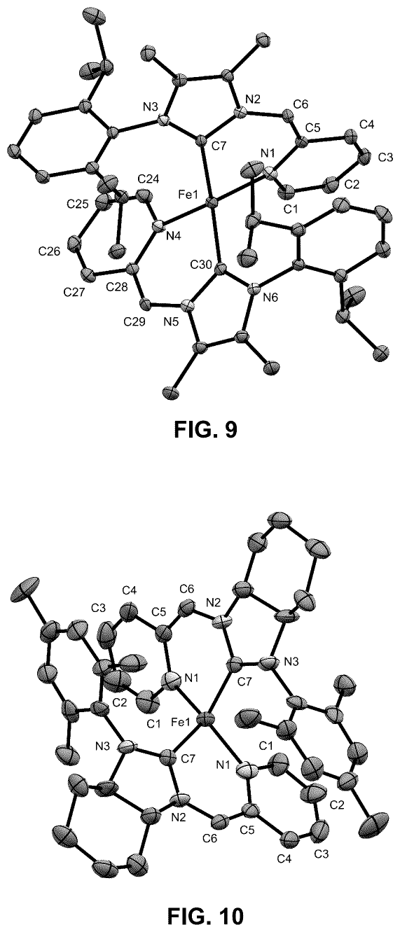

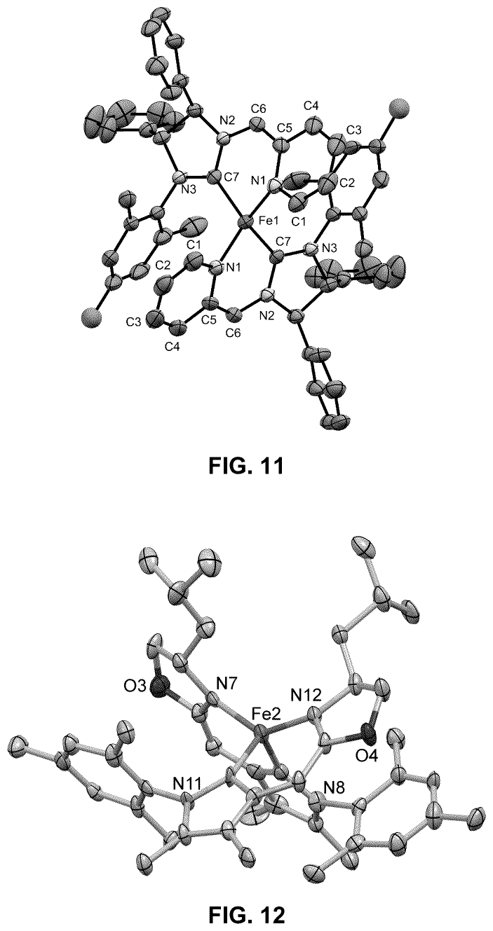

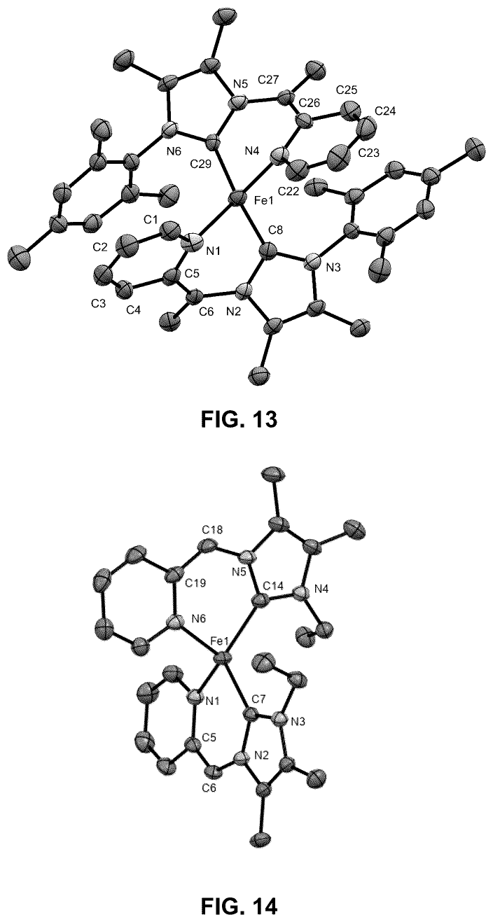

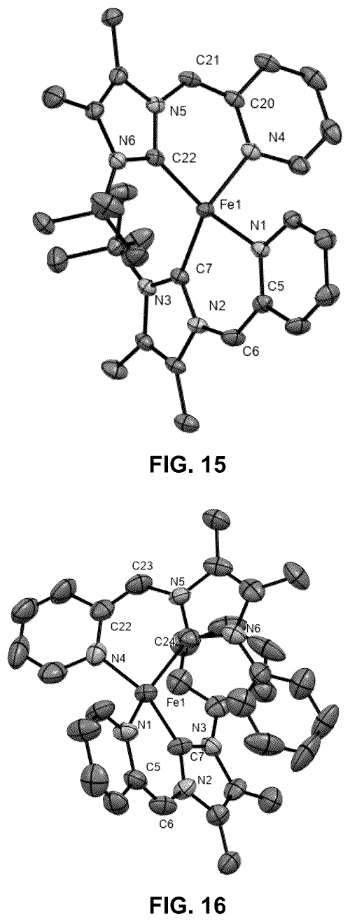

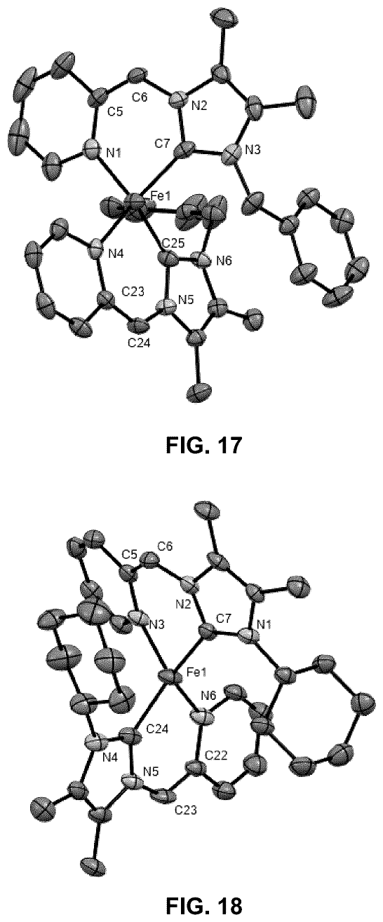

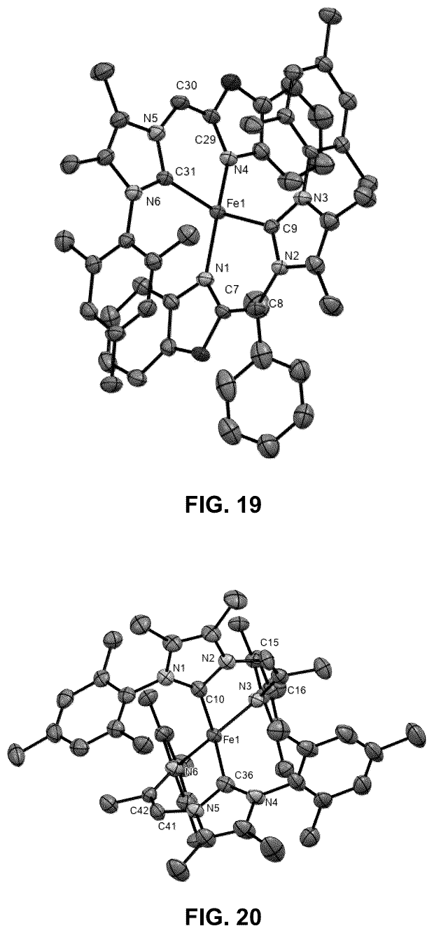

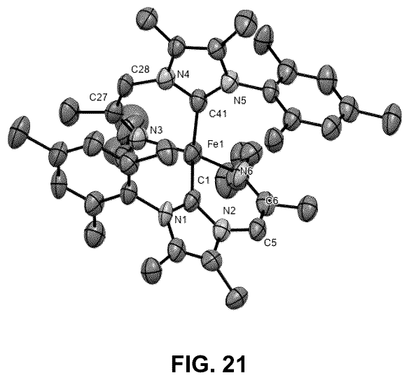

The X-ray diffraction data were collected on a Bruker Kappa Apex II/Photon II diffractometer with graphite-monochromated Mo Kα radiation (λ=0.71073 Å) at 150 K controlled by an Oxford Cryostream 700 series low-temperature system and processed with the Bruker Apex 3 software package (Apex2 Software Package; Bruker AXS Inc.: Madison, WI, 2013). The structures were solved by direct methods and refined using SHELX-2016 software package (Sheldrick G. M. Crystal structure refinement with SHELXL. Acta Crystallogr., Sect. C: Struct. Chem. 2015, C71, 3; http://shelx.uni-ac.gwdg.de/SHELX/index.php. (accessed on Jan. 30, 2017)). All non-hydrogen atoms were refined anisotropically. The diffuse residual electron density of lattice solvent molecules in the lattices of [9] and [10] was removed with the SQUEEZE function of PLATON (Spek A. L. J. Appl. Crystallogr. 2003, 36, 7) and was not included in the formula or the refinement. X-ray structure of complexes [2]-[22] are reported in FIGS. 1-21. Selected crystallographic data are summarized in Tables 1-5.

TABLE 1
Selected crystallographic data for compounds [2], [3•C₄H₁₀O],
[4], [5], and [6]

| | 2 | 3•C₄H₁₀O | 4 | 5 | 6 |
|---|---|---|---|---|---|
| Empirical | C₃₀H₂₄N₆Fe | C₄₆H₅₈N₆FeO | C₂₆H₃₂N₆Fe | C₃₆H₄₀N₆Fe | C₄₀H₄₄N₆Fe |
| formula | | | | | |
| FW (g · mol⁻¹) | 524.40 | 766.83 | 484.42 | 612.59 | 664.66 |
| Crystal system | Triclinic | Orthorhombic | Monoclinic | Monoclinic | Triclinic |
| Space Group | P-1 | Pbca | P2₁/n | P2₁/c | |
| Z | 2 | 8 | 4 | 4 | 2 |
| a (Å) | 10.58(3) | 11.203(3) | 9.201(2) | 13.542(6) | 8.8585(9) |
| b (Å) | 11.108(18) | 23.766(7) | 18.759(6) | 9.618(4) | 11.8948(12) |
| c (Å) | 11.59(3) | 31.176(9) | 14.696(4) | 23.983(10) | 18.4729(17) |
| α (deg) | 110.06(8) | 90 | 90 | 90 | 107.170(4) |
| β (deg) | 101.65(9) | 90 | 95.671(9) | 101.353(9) | 94.597(4) |
| γ (deg) | 96.86(8) | 90 | 90 | 90 | 105.980(4) |
| V (Å³) | 1226(5) | 8301(4) | 2524.1(12) | 3063(2) | 1760.1(3) |
| D_{calcd,} (g · | 1.420 | 1.227 | 1.275 | 1.329 | 1.254 |
| cm⁻³) | | | | | |
| μ (mm⁻¹) | 0.647 | 0.405 | 0.622 | 0.529 | 0.465 |
| F(000) | 544 | 3280 | 1024 | 1296 | 704 |
| no. of obsd | 3237 | 4764 | 4831 | 4183 | 6640 |
| reflns | | | | | |
| no. of params | 335 | 501 | 304 | 395 | 434 |
| refnd | | | | | |
| goodness of fit | 1.022 | 1.049 | 1.095 | 1.032 | 1.073 |
| R₁ (I > 2σ) | 0.0823 | 0.0843 | 0.0268 | 0.0537 | 0.0397 |
| wR₂ | 0.2188 | 0.2077 | 0.0784 | 0.1301 | 0.1176 |

TABLE 2:
Selected crystallographic data for compounds [7•C₅H₁₂],
[8•C₇H₈], [9], [10] and [11]

| | 7•C₅H₁₂ | 8•C₇H₈ | 9 | 10 | 11 |
|---|---|---|---|---|---|
| Empirical | C₄₃H₅₂N₆Fe | C₅₁H₆₀N₆Fe | C₆₀H₅₂FeN₆ | C₄₆H₅₆N₆Fe | C₄₄H₅₂N₆Fe |
| formula | | | | | |
| FW (g · mol⁻¹) | 708.75 | 812.90 | 912.92 | 748.81 | 720.76 |
| Crystal | Triclinic | Monoclinic | Monoclinic | Triclinic | Monoclinic |
| system | | | | | |
| Space Group | P-1 | P2₁/c | P2₁/n | P-1 | C2 |
| Z | 2 | 4 | 8 | 2 | 4 |
| a (Å) | 10.933(10) | 19.783(5) | 15.250(5) | 11.293(6) | 21.406(14) |
| b (Å) | 11.269(10) | 12.161(3) | 29.324(11) | 12.732(6) | 10.270(7) |
| c (Å) | 17.237916) | 19.892(5) | 24.658(9) | 16.879(7) | 17.354(12) |
| α (deg) | 87.02(3) | 90 | 90 | 97.395(14) | 90 |
| β (deg) | 74.18(3) | 110.403(9) | 90 | 95.556(14) | 105.20(2) |
| γ (deg) | 69.78(3) | 90 | 90 | 113.908(13) | 90 |
| V (Å3) | 1915(3) | 4486(2) | 11027(7) | 2170.3(17) | 3720(4) |
| D_{calcd}, (g · | 1.229 | 1.204 | 1.100 | 1.146 | 1.287 |
| cm⁻³) | | | | | |
| μ (mm⁻¹) | 0.432 | 0.377 | 0.314 | 0.384 | 0.446 |
| F(000) | 756 | 1736 | 3840 | 800 | 1536 |
| no. of obsd | 3302 | 7299 | 13208 | 6119 | 4714 |
| reflns | | | | | |
| no. of params | 461 | 555 | 1188 | 490 | 467 |
| refnd | | | | | |
| goodness of | 0.980 | 1.012 | 0.993 | 1.030 | 1.118 |
| fit | | | | | |
| R₁ (I > 2σ) | 0.1292 | 0.0663 | 0.1665 | 0.0589 | 0.0875 |
| wR₂ | 0.3665 | 0.1574 | 0.3819 | 0.1527 | 0.1787 |

TABLE 3
Selected crystallographic data for compounds [12], [13] and [14].

| | 12 | 13 | 14 |
|---|---|---|---|
| Empirical formula | C₆₀H₅₆N₆Fe | C₄₄H₆₀FeN₆O₂ | C₄₂H₄₈N₆Fe |
| FW (g · mol⁻¹) | 916.95 | 760.83 | 692.71 |
| Crystal system | Monoclinic | Triclinic | Triclinic |
| Space Group | I2 | P1 | P-1 |
| Z | 2 | 2 | 4 |
| a (Å) | 11.602(5) | 12.313(19) | 8.812(5) |
| b (Å) | 9.775(5) | 12.570(18) | 18.765(11) |
| c (Å) | 22.430(11) | 15.85(2) | 23.457(14) |
| α (deg) | 90 | 95.33(4) | 74.837(19) |
| β (deg) | 100.08(4) | 111.95(3) | 84.66(2) |
| γ (deg) | 90 | 110.14(3) | 77.248(18) |
| V (Å3) | 2505(2) | 2066(5) | 3649(4) |
| D_{calcd,} (g · cm⁻³) | 1.216 | 1.223 | 1.261 |
| μ (mm⁻¹) | 0.346 | 0.408 | 0.452 |
| F(000) | 968 | 816 | 1472 |
| no. of obsd reflns | 3920 | 6440 | 6752 |
| no. of params refnd | 306 | 929 | 908 |
| goodness of fit | 1.010 | 0.909 | 0.988 |
| R₁ (I > 2σ) | 0.0582 | 0.0889 | 0.0964 |
| wR₂ | 0.1284 | 0.1559 | 0.2286 |

TABLE 4
Selected crystallographic data for compounds
[15], [16], [17], [18] and[19].

| | 15•C₄H₈O | 16 | 17 | 18 | 19 |
|---|---|---|---|---|---|
| Empirical formula | C₃₀H₄₀N₆Fe₀ | C₃₀H₄₀N₆Fe | C₃₄H₃₂N₆Fe | C₃₆H₃₆N₆Fe | C₃₄H₄₄N₆Fe |
| FW (g · mol⁻¹) | 556.53 | 540.53 | 580.50 | 608.56 | 592.60 |
| Crystal system | Triclinic | Monoclinic | Orthorhombic | Triclinic | Monoclinic |
| Space Group | P-1 | P2₁/n | Pbca | P-1 | P2₁/n |
| Z | 2 | 4 | 8 | 2 | 4 |
| a (Å) | 8.275(2) | 9.884(4) | 17.01(2) | 10.319(3) | 11.479(11) |
| b (Å) | 11.789(3) | 18.392(8) | 17.01(2) | 11.059(3) | 17.125(15) |
| c (Å) | 15.453(4) | 16.076(7) | 20.54(2) | 14.190(3) | 15.798(15) |
| α (deg) | 79.789(8) | 90 | 90 | 77.342(8) | 90 |
| β (deg) | 76.709(8) | 100.084(13) | 90 | 78.411(8) | 99.59 |
| γ (deg) | 75.484(8) | 90 | 90 | 89.077(9) | 90 |
| V (Å3) | 1408.9(7) | 2877(2) | 5943(11) | 1547.1(7) | 3062(5) |
| D_{calcd}, (g · cm⁻³) | 1.312 | 1.248 | 1.298 | 1.306 | 1.285 |
| μ (mm⁻¹) | 0.569 | 0.553 | 0.541 | 0.523 | 0.526 |
| F(000) | 592 | 1152 | 2432 | 640 | 1264 |
| no. of obsd reflns | 6585 | 6605 | 6781 | 13074 | 6956 |
| no. of params refnd | 349 | 344 | 374 | 783 | 375 |
| goodness of fit | 1.068 | 1.107 | 1.255 | 0.953 | 1.008 |
| R₁ (I > 2σ) | 0.0447 | 0.0570 | 0.1893 | 0.0332 | 0.0957 |
| wR₂ | 0.1270 | 0.1900 | 0.4782 | 0.0661 | 0.2152 |

TABLE 5
Selected crystallographic data for compounds [20], [21], and [22].

| | 20•C₇H₈ | 21 | 22 |
|---|---|---|---|
| Empirical formula | C₅₁H₅₂N₆FeO₂ | C₅₂H₆₄N₆Fe | C₄₂H₅₀N₆Fe |
| FW (g · mol⁻¹) | 836.83 | 828.94 | 694.73 |
| Crystal system | Monoclinic | Monoclinic | Triclinic |
| Space Group | P2₁/n | P2₁/n | P-1 |
| Z | 4 | 4 | 2 |
| a (Å) | 12.537(7) | 11.374(7) | 11.288(9) |
| b (Å) | 19.126(11) | 22.981(14) | 11.547(9) |
| c (Å) | 19.650(11) | 19.957(13) | 17.151(14) |
| α (deg) | 90 | 90 | 89.480(18) |
| β (deg) | 100.338(17) | 90.886(18) | 76.401(17 |
| γ (deg) | 90 | 90 | 69.646(17) |
| V (Å3) | 4635(4) | 5216(6) | 2031(3) |
| D_{calcd}, (g · cm⁻³) | 1.199 | 1.056 | 1.136 |
| μ (mm⁻¹) | 0.370 | 0.326 | 0.406 |
| F(000) | 1768 | 1776 | 740 |
| no. of obsd reflns | 10583 | 12078 | 9755 |
| no. of params refnd | 553 | 551 | 444 |
| goodness of fit | 1.045 | 1.022 | 0.990 |
| R₁ (I > 2σ) | 0.0931 | 0.1128 | 0.1298 |
| wR₂ | 0.2751 | 0.3070 | 0.3701 |

### Catalytic Hydrosilylation of Ketones Using Complexes [1]-[22]

### General Experimental Procedure for Initial Catalytic Performance Evaluation

In a nitrogen glovebox, catalyst (1.75 umol, 0.05 mol %), acetophenone (0.41 mL, 3.5 mmol) and mesitylene (35 uL, 0.25 mmol, internal standard) were charged in a 2-dram borosilicate glass vial and stirred for 10 minutes to solubilize the catalyst. PMHS (0.53 mL, 8.75 mmol) was then added to the mixture. The reaction was stirred at ambient temperature for the indicated time (Tables 6 and 7). An NMR sample was prepared using 0.1 mL of the reaction mixture in 0.4 ml of CDCl₃. The conversions of the reactions were determined by ¹H NMR signals of the products compared to internal standard. The results are summarized in Tables 6 and 7.

TABLE 6
Catalytic Activities of Complexes [1]-[14] toward the hydrosilylayion of
acetophenone^{a}

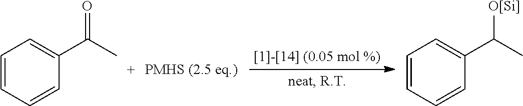

| Entry | Catalyst | Time (min) | Conversion (%) | ee (%)^{b} |
|---|---|---|---|---|
| 1 | 1 | 5 | 12 | — |
| 2 | 1 | 120 | >99 | — |
| 3 | 2 | 120 | 75 | — |
| 4 | 3 | 120 | 96 | — |
| 5 | 4 | 120 | 73 | — |
| 6 | 5 | 120 | >99 | — |
| 7 | 5 | 5 | <5 | — |
| 8 | 6 | 5 | >99 | — |
| 9 | 7 | 5 | 31 | — |
| 10 | 8 | 5 | >99 | — |
| 11 | 9 | 120 | 87 | — |
| 12 | 10 | 5 | >99 | — |
| 13 | 11 | 180 | >99 | — |
| 14 | 12 | 180 | >99 | — |
| 15 | 13 | 5 | >99 | — |
| 16 | 14 | 120 | 50 | — |
| 17^{c} | 11 | 180 | >99 | 4 |
| 18^{c} | 12 | 180 | >99 | 24 |
| 19^{c} | 13 | 5 | >99 | 0 |

^{a}General reaction conditions: acetophenone (3.5 mmol) in PMHS (8.75 mmol, 2.5 equiv.), ambient temperature. The conversions are based on the ¹H NMR integrations using mesitylene as the internal standard.
^{b}ee were determined by GC analysis.
^{c}(EtO)₂MeSiH (8.75 mmol, 2.5 eq.) was used.

TABLE 7
Catalytic Activities of Complexes [15]-[22] toward the hydrosilylation of
acetophenone^{a}

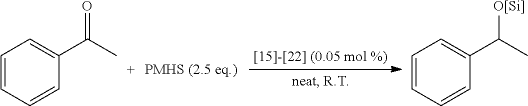

| Entry | Catalyst | Time (min) | Conversion (%) | ee (%) |
|---|---|---|---|---|
| 1 | 15 | 5 | 85 | — |
| 2 | 15 | 120 | >99 | — |
| 3 | 16 | 5 | 12 | — |
| 4 | 16 | 120 | >99 | — |
| 5 | 17 | 5 | 30 | — |
| 6 | 17 | 120 | >99 | — |
| 7 | 18 | 5 | 30 | — |
| 8 | 18 | 120 | >99 | — |
| 9 | 19 | 5 | 40 | — |
| 10 | 19 | 60 | >99 | — |
| 11 | 20 | 5 | 15 | — |
| 12 | 20 | 120 | 85 | — |
| 13 | 21 | 5 | 20 | — |
| 14 | 21 | 120 | >99 | — |
| 15 | 22 | 5 | >99 | — |

^{a}General reaction conditions: acetophenone (3.5 mmol) in PMHS (8.75 mmol, 2.5 equiv.), ambient temperature. The conversions are based on the ¹H NMR integrations using mesitylene as the internal standard.

### Catalytic Hydrosilylation of Acetophenone With Complex [6] Using Various Silanes

The catalytic activity of complex [6] toward the hydrosilylation of acetophenone using various silanes was determined and the results are reported in Table 8.

TABLE 8
Scope of silanes^{a}

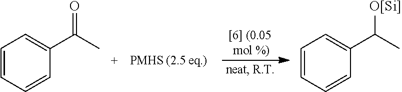

| Entry | Silane | Time (min) | Conversion (%) |
|---|---|---|---|
| 1 | PMHS (2.5 eq.) | 5 | >99 |
| 2 | TMDS (1.25 eq.) | 5 | >99 |
| 3 | (EtO)₂MeSiH (2.5 eq.) | 5 | >99 |
| 4 | PhSiH₃ (1 eq.) | 5 | >99 |
| 5 | Ph₂SiH₂ (1 eq.) | 5 | >99 |

^{a}General reaction conditions: acetophenone (3.5 mmol), silane, ambient temperature. The conversions are based on the ¹H NMR inegrations using mesitylene as the internal standard.

### Catalytic Hydrosilylation of Various etones With coCmplex [6]

In a nitrogen glovebox, complex 6 (1.1 mg, 1.75 μmol, 0.05 mol %) and ketone (3.5 mmol) were charged in a 2-dram borosilicate glass vial and stirred for 10 minutes to solubilize the catalyst. PMHS (0.53 mL, 8.75 mmol) was then added to the mixture. The reaction was left stirring at ambient temperature for the indicated time (Table 9). The vial was then removed from the glovebox and the contents were hydrolyzed with 2 M NaOH (2 mL) and stirred overnight. The mixture was extracted with diethyl ether (3×10 mL). The combined organic layer was washed with brine (2×10 mL), dried over MgSO₄, and concentrated under reduced pressure to afford the desired alcohol.

TABLE 9
Substrate scope of the iron-catalyzed hydrosilylation of ketone^{a}

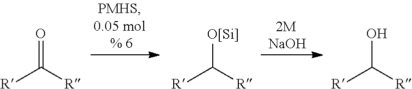

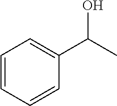

5 min, 99%

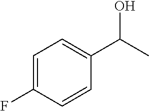

5 min, 99%

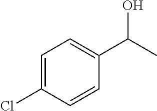

10 min, 99%

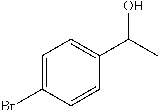

10 min, 99%^{b}

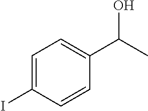

10 min, 99%^{b}

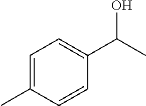

5 min, 99%^{b}

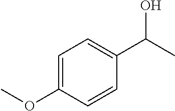

5 min, 98%^{b}

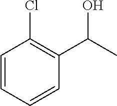

10 min, 98%

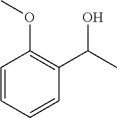

5 min, 98%

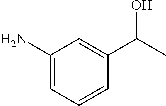

30 min, 95%^{b}

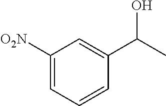

30 min, 55%^{b}

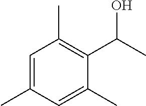

5 min, 99%

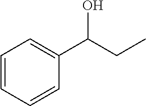

5 min, 98%

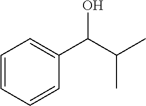

5 min, 99%

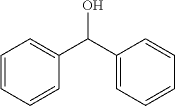 [SMILES: OC(c1ccccc1)c1ccccc1]

30 min, 95%^{b}

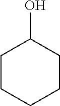

5 min, 99%

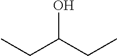

10 min, 99%

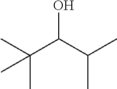

30 min, 990%

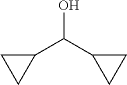

30 min, 98%

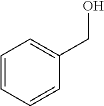

5 min, 99%

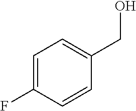

5 min, 99%
^{a}Percent conversion as determined by ¹H NMR spectroscopy.
^{b}for solid substrates, 0.3 mL of benzene were added.

### Determination of Turnover Frequency TOF at 30 Seconds

In a nitrogen glovebox, complex [6] (1.1 mg, 1.75 μmol, 0.05 mol %), acetophenone (0.41 mL, 3.5 mmol) and mesitylene (35 μL, 0.25 mmol, internal standard) were charged in a 2-dram borosilicate glass vial and stirred for 10 minutes to solubilize the catalyst. PMHS (0.53 mL, 8.75 mmol) was then added to the mixture. The reaction was stirred at room temperature for 30 seconds. An NMR sample was prepared using 0.1 mL of the reaction mixture in 0.4 mL of CDCl₃. ¹H NMR analysis of the reaction revealed 60% conversion, giving the TOF (30 s) of 40 s⁻¹.

### Determination of Turnover Number TON From a Multidose Experiment

In a nitrogen glovebox, a round bottom flask was charged with complex [6] (1.0 mg, 1.50 μmol, 1 equiv.) and acetophenone (4.7 mL, 40 mmol, 26,700 equiv.) were added and the mixture was stirred for 2 min. PMHS (3.6 mL, 60 mmol, 40,000 equiv.) was then added to the mixture. The reaction was stirred at room temperature for 2 hours. A second batch of acetophenone (4.7 mL, 40 mmol, 26,700 equiv.) and PMHS (3.6 mL, 60 mmol, 40,000 equiv.) was added and the reaction was stirred at room temperature for 2 hours. A third batch of acetophenone (4.7 mL, 40 mmol, 26,700 equiv.) and PMHS (3.6 mL, 60 mmol, 40,000 equiv.) was added and the reaction was stirred at room temperature for 24 hours. A ¹H NMR spectrum of the mixture revealed complete conversion of the substrate into silyl ether, giving the total turnover number of 80,000.

### Wax-Protected Iron Catalyst—Preparation and Catalytic Evaluation

Dispersing into Paraffin wax has shown to be a good strategy to protect air-sensitive catalysts (Fang, Y. et al. Applied Catalysis A: General, 2002, 235, 33-38; Frankowski, K. J., Taber, D. F. J. Org. Chem. 2003, 68, 6047-6048). The dispersed catalyst-Paraffin mixture can be stored and handled in ambient air. A wax-protected catalyst [6] was prepared and was stored under ambient conditions for 8 months with only a slight loss of catalytic activity.

The solid Paraffin wax was purchased from ParoWax (density ˜0.75 g/cm³, melting range 58-60° C.) and was dried at 90° C. under vacuum (20 mtorr) for 24 h prior to use. In a nitrogen-filled glovebox, a 20 mL flask was charged with Paraffin wax (2 g), [6] (50 mg, 0.075 mmol), and a stirring bar and then sealed. With vigorous stirring at 60° C., a homogeneously dispersed wax-protected catalyst was obtained in 15 min and allowed to cool down and solidify. The resulting wax-protected [6] was then stored in amber vials under ambient atmosphere.

The catalysis was carried out under air-free conditions using standard Schlenk techniques. The wax-protected [6] was weighed (160 mg, 0.006 mmol of [6]) in air and transferred into a 10 mL Schlenk tube, which was then purged under vacuum at ambient temperature for 2 h and refilled with N₂. Then acetophenone (1.44 g, 12 mmol) was added via syringe and the Schlenk tube was heated at 60° C. to melt the wax. After stirring for 10 min at 60° C. the silane PMHS was added (1.8 g, 30 mmol) to the homogeneous mixture. The system was allowed to stir without a heating bath for 1 hour. The Schlenk tube was then opened to air and the contents were hydrolyzed with 2 M NaOH (20 mL) and stirred overnight. The mixture was extracted with diethyl ether (3×20 mL). The combined organic layer was washed with brine (2×15 mL), dried over MgSO₄, and concentrated under reduced pressure to afford the 1-phenylethanol as the product.

The catalytic performance of wax-protected [6] stored in air for various durations was summarized in Table 10. These results demonstrate that the wax-protected [6] can be used toward carbonyl reduction with PMHS using the standard Schlenk techniques without the need of an inert atmosphere glovebox. The wax-protected [6] remains active after stored in ambient air for 8 months. Despite the slight activity loss due to the 8 months of storage in air, dispersing the catalysts into Paraffin wax is a viable method for catalysis, which eliminates the requirement of gloveboxes for the end user of the catalysts.

TABLE 10
Catalytic performance of the wax-protected [6] toward the
hydrosilylation of acetophenone using PMHS^{a}

| | | Catalyst | Reaction | |
|---|---|---|---|---|
| | Duration | loading | time | Conversion |
| Entry | of stage in air | (mol %) | (min) | (%) |
| 1 | 0 | 0.05ª | 5 | >99 |
| 2 | 24 h | 0.05ª | 5 | 30 |
| 3 | 24 h | 0.05ª | 30 | >99 |
| 4 | 3 months | 0.05ª | 60 | 40 |
| 5 | 3 months | 0.1b | 60 | >99 |
| 6 | 8 months | 0.05ª | 60 | 30 |
| 7 | 8 months | 0.1^{b} | 120 | >99 |

^{a}General reaction conditions: wax-protected [6] (160 mg), acetophenone (1.44 g, 12.5 mmol), PMHS (1.8 g, 30 mmol), ambient temperature.
^{b}wax-protected [6] (320 mg), acetophenone (1.44 g,12.5 mmol), PMHS (1.8 g, 30 mmol), ambient temperature. The conversions are based on the ¹H NMR integrations using mesitylene as the internal standard.

1. An iron-based complex of formula (IA) or (IB) or a solvate thereof 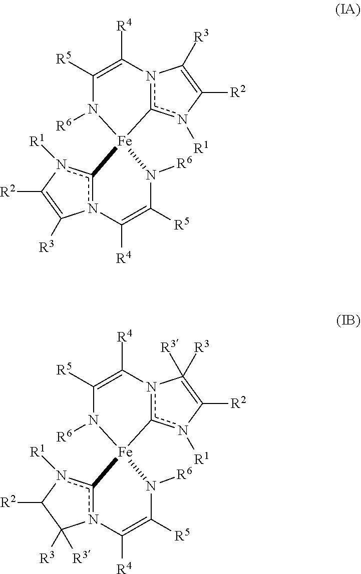
wherein:
the chain 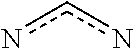
comprise one single bond and one double bond;
R¹ represents a linear or branched C₁-C₁₈alkyl, linear or branched C₂-C₁₈alkenyl, C₃-C₁₀cycloalkyl, C₃-C₁₀cycloalkenyl or C₆-C₁₄aryl, where the alkyl, alkenyl, cycloalkyl, cycloalkenyl and aryl are optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
R², R²′, R³ and R³′, which are identical or different, represent H, a linear or branched C₁-C₁₀alkyl, linear or branched C₂-C₁₀alkenyl, C₃-C₁₀cycloalkyl, C₃-C₁₀cycloalkenyl or a C₆-C₁₄aryl; where the alkyl, alkenyl, cycloalkyl, cycloalkenyl, and aryl are optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, OCF₃, and CF₃; provided that in formula (IA) at least one of R² and R³ is different than H;
or R² and R³ are bound to form a 5-or 6-membered cycloalkyl group containing the carbon atom bearing R² and the carbon atom bearing R³; where the 5-or 6-membered cycloalkyl is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
R⁴represents H or a linear or branched C₁-C₁₈alkyl, C₃-C₆cycloalkyl or C₆-C₁₀aryl;
R⁵ and R⁶ , which are identical or different, represent a linear or branched C₁-C₁₈alkyl, linear or branched C₂-C₁₈alkenyl, C₃-C₁₀cycloalkyl, C₃-C₁₀cycloalkenyl or C₆-C₁₄aryl, where the alkyl, alkenyl, cycloalkyl, cycloalkenyl and aryl are optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
or R⁵ and R⁶ are bound to form a saturated or unsaturated 5-or 6-membered heterocyclic group containing the nitrogen atom bearing R⁶ and the carbon atom bearing R⁵ , where the 5-or 6-membered heterocyclic group optionally includes 1 or 2 additional heteroatoms selected from N, O and S, and where the 5-or 6-membered heterocyclic group is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃;
or R⁵ and R⁶ are bound to form a saturated or partially unsaturated 7-to 10-membered fused heterocyclic group containing the nitrogen atom bearing R⁶ and the carbon atom bearing R⁵ where the 7-to 10-membered fused heterocyclic group optionally includes 1 or 2 additional heteroatoms selected from N, O and S, and where the 7-to 10-membered fused heterocyclic group is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷, C(O)OR⁷, C(O)NH₂, C(O)NHR⁷, C(O)NR⁷R⁷, tetrahydrofuran-2-yl, tetrahydrofuran-3-yl, NO₂, CN, NH₂, OCF₃, and CF₃;
R⁷and R⁷′, which are identical or different, represent a C₁-C₆alkyl, C₁-C₆cycloalkyl, C₂-C₆alkenyl, C₂-C₆cycloalkenyl, or C₆-C₁₄aryl.

2. The iron-based complex of claim 1, wherein R² and R³, which are identical or different, represent H, a linear or branched C₁-C₆alkyl, or a C₆aryl, where the alkyl and aryl are optionally substituted with one or more substituents selected from the group consisting of halogen, linear or branched C₁-C₆alkyl, C₆-C₁₀aryl, vinyl, NO₂, and CF₃, provided that in the formula (IA) at least one of R² and R³ is different than H.

3. The iron-based complex of claim 1, wherein R² and R³, which are identical or different, represent H, Me, Et, or Ph, provided that in the formula (IA) at least one of R² and R³ is different than H.

4. The iron-based complex of claim 1, wherein R² is H and R³ represents a linear or branched C₁-C₆alkyl or a C₆aryl, where the alkyl and aryl are optionally substituted with one or more substituents selected from the group consisting of halogen, linear or branched C₁-C₆alkyl, C₆-C₁₀aryl, linear or branched C₁-C₆alkoxy, OCF₃, and CF₃.

5. The iron-based complex of claim 4, wherein R³ is H, Me, Et, or Ph.

6. The iron-based complex of claim 1, wherein R² and R³ are identical and represent a linear or branched C₁-C₆alkyl, or a C₆aryl, where the alkyl and aryl are optionally substituted with one or more substituents selected from the group consisting of halogen, linear or branched C₁-C₆alkyl, C₆-C₁₀aryl, linear or branched C₂-C₆alkenyl, linear or branched C₁-C₆alkoxy, OCF₃ and CF₃.

7. The iron-based complex of claim 1, wherein R² and R³ are identical and represent Me, Et, or Ph.

8. The iron-based complex of claim 1, wherein R² and R³ are bound to form a 5-or 6-membered cycloalkyl group optionally substituted with 1 to 4 substituents selected from the group consisting of halogen, linear or branched C₁-C₆alkyl, C₆-C₁₀aryl, linear or branched C₂-C₆alkenyl, linear or branched C₁-C₆alkoxy, OCF₃, and CF₃.

9. The iron-based complex of any one of claims 1 to 8, wherein the complex is of formula (IA).

10. The iron-based complex of any one of claims 1 to 8, wherein the complex is of formula (IB) and R²′ and R³′ are identical or different and represent H, a linear or branched C₁-C₆alkyl or a C₆aryl, where the alkyl and aryl are optionally substituted with one or more substituents selected from the group consisting of halogen, linear or branched C₁-C₆alkyl, C₆-C₁₀aryl, linear or branched C₂-C₆alkenyl, linear or branched C₁-C₆alkoxy, OCF₃, and CF₃.

11. The iron-based complex of any one of claims 1 to 8, wherein the complex is of formula (IB) and R²′ and R³′ are identical or different and represent H, Me, Et, or Ph.

12. The iron-based complex of any one of claims 1 to 8, wherein the complex is of formula (IB) and R²′ and R³′ are identical and represent H, Me, Et, or Ph.

13. The iron-based complex of any one of claims 1 to 8, wherein the complex is of formula (IB), R² and R³ are identical and represent Me, Et, or Ph, and R²′ and R³′ are H.

14. The iron-based complex of any one of claims 1 to 8, wherein the complex is of formula (IB) where R²′ and R³′ represent H and R² and R³ are bound to form a 5-or 6-membered cycloalkyl group optionally substituted with 1 to 4 substituents selected from the group consisting of halogen, linear or branched C₁-C₆alkyl, C₆-C₁₀aryl, linear or branched C₂-C₆alkenyl, linear or branched C₁-C₆alkoxy, OCF₃, and CF₃.

15. The iron-based complex of claim 14, wherein R² and R³ are bound to form a cyclohexyl group.

16. The iron-based complex of any one of claims 1 to 15, wherein R¹ represents a linear or branched C₁-C₆alkyl, a C₃-C₆cycloalkyl or C₆aryl, where the C₃-C₆cycloalkyl and C₆aryl are optionally substituted with one or more substituents selected from the group consisting of halogen, linear or branched C₁-C₆alkyl, C₆-C₁₀aryl, linear or branched C₂-C₆alkenyl, linear or branched C₁-C₆alkoxy, OCF₃, and CF₃.

17. The iron-based complex of any one of claims 1 to 16, wherein R¹ represents a linear or branched C₁-C₆alkyl, a C₃-C₆cycloalkyl or C₆aryl, where the C₃-C₆cycloalkyl and C₆aryl are optionally substituted with 1 to 3 linear or branched C₁-C₆alkyl.

18. The iron-based complex of any one of claims 1 to 17, wherein R¹ represents a linear or branched C₁-C₆alkyl, or C₆aryl, where the C₆aryl is optionally substituted with 1 to 3 linear or branched C₁-C₄alkyl.

19. The iron-based complex of any one of claims 1 to 18, wherein R¹ represents Me, Et, nPr, iPr, nBu, sBu, tBu, Cy, Ph, Bn, C₆H₄Me, C₆H₃(Me)₂, C₆H₂(Me)₃, C₆H₄(iPr), C₆H₃(iPr)₂, C₆H₂(iPr)₃, C₆H₄(tBu), C₆H₃(tBu)₂, or C₆H₂(tBu)₃.

20. The iron-based complex of any one of claims 1 to 19, wherein R¹ represents Et, Ph, tBu, C₆H₂(Me)₃, or C₆H₃(iPr)₂.

21. The iron-based complex of any one of claims 1 to 20, wherein R⁴ represents H or a linear or branched C₁-C₄alkyl.

22. The iron-based complex of any one of claims 1 to 21, wherein R⁴ represents H or Me.

23. The iron-based complex of any one of claims 1 to 22, wherein R⁴ represents H.

24. The iron-based complex of any one of claims 1 to 23, wherein R⁵ and R⁶ are bound to form a saturated or unsaturated 5-or 6-membered heterocyclic group containing the nitrogen atom bearing R⁶ and the carbon atom bearing R⁵ , where the 5-or 6-membered heterocyclic group optionally includes 1 or 2 additional heteroatoms selected from N, O and S, and where the 5-or 6-membered heterocyclic group is optionally substituted with 1 to 4 substituents selected from the group consisting of halogen, linear or branched C₁-C₆alkyl, C₆-C₁₀aryl, linear or branched C₂-C₆alkenyl, linear or branched C₁-C₆alkoxy, OCF₃ and CF₃.

25. The iron-based complex of any one of claims 1 to 24, wherein R⁵ and R⁶ are bound to form a saturated or unsaturated 5-or 6-membered heterocyclic group containing the nitrogen atom bearing R⁶ and the carbon atom bearing R⁵ , where the 5-or 6-membered heterocyclic group optionally includes 1 or 2 additional heteroatoms selected from N, O and S, and where the 5-or 6-membered heterocyclic group is optionally substituted with a linear or branched C₁-C₆alkyl.

26. The iron-based complex of any one of claims 1 to 23, wherein R⁵ and R⁶ are bound to form a saturated or partially unsaturated 9-or 10-membered fused heterocyclic group containing the nitrogen atom bearing R⁶ and the carbon atom bearing R⁵ , where the 9-or 10-membered fused heterocyclic group optionally includes 1 or 2 additional heteroatoms selected from N, O and S, and where the 9-or 10-membered fused heterocyclic group is optionally substituted with a linear or branched C₁-C₆alkyl.

27. The iron-based complex of any one of claims 1 to 26, wherein the complex is of formula (IA′) or (IB′) 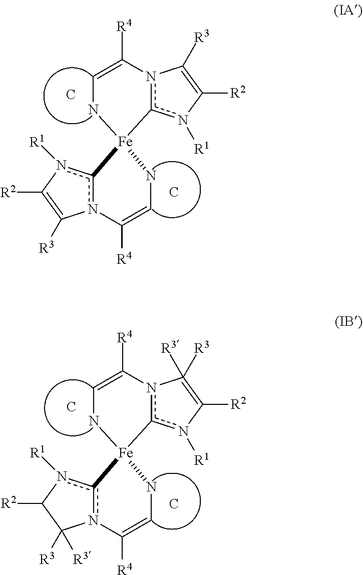
wherein 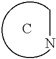
represents a moiety selected from the group consisting of 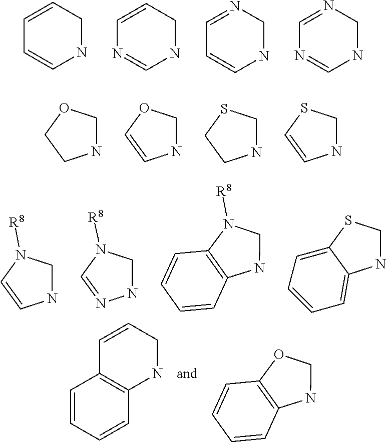
wherein R⁸ is a linear or branched C₁-C₆alkyl or C₆-C₁₄aryl and the moiety is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷′, tetrahydrofuran-2-yl, tetrahydrofuran-3-yl, NO₂, CN, NH₂, OCF₃, and CF₃, wherein R⁷and R⁷′ are as defined in claim 1.

28. The iron-based complex of claim 27, wherein the moiety is substituted with a linear or branched C₁-C₆alkyl.

29. The iron-based complex of claim 27, wherein the moiety is unsubstituted.

30. The iron-based complex of claim 1, wherein the complex is selected from the group consisting of

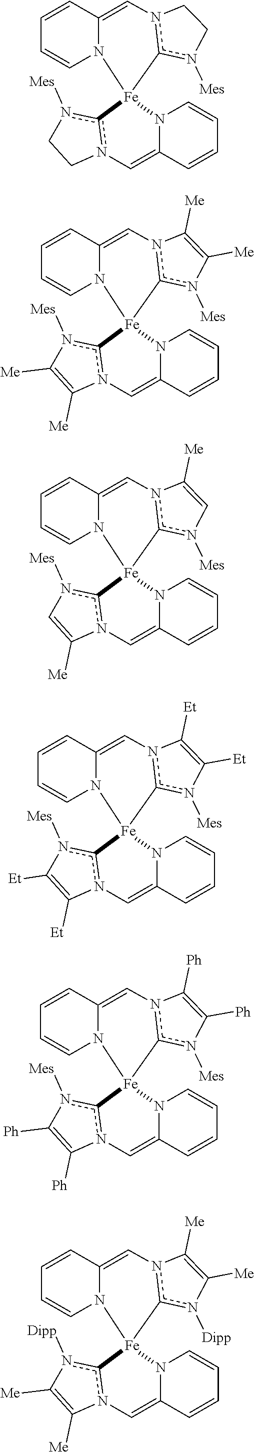

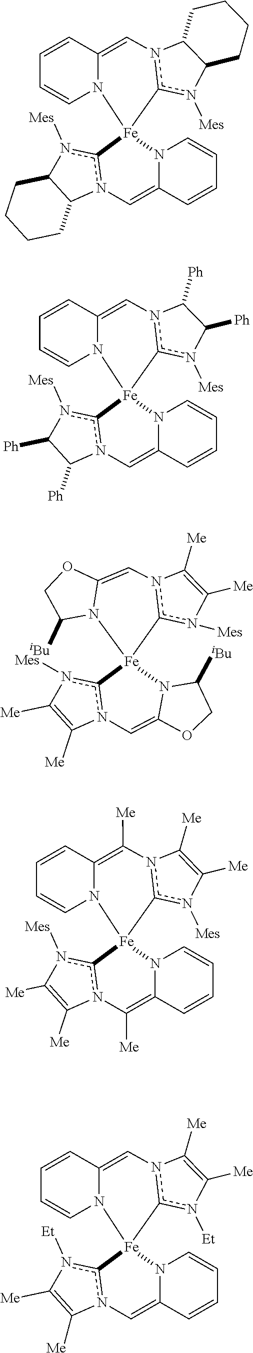

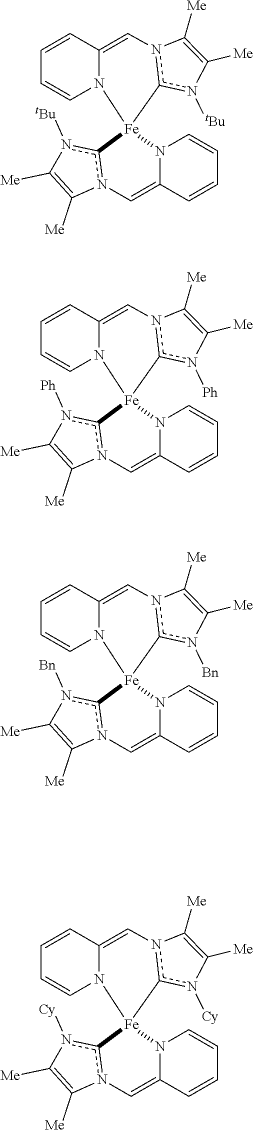

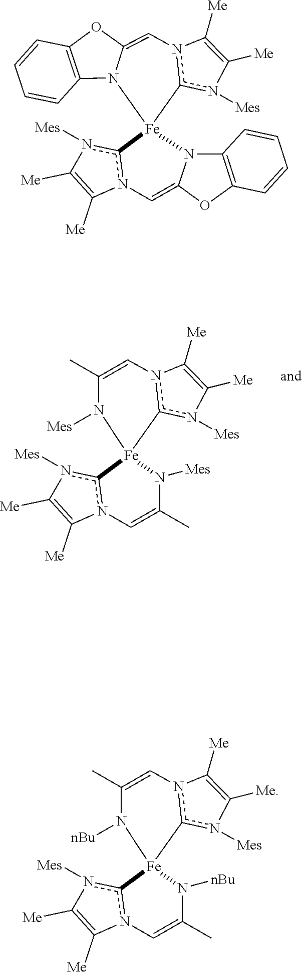

31. The iron-based complex of claim 1, wherein the complex is selected from the group consisting of

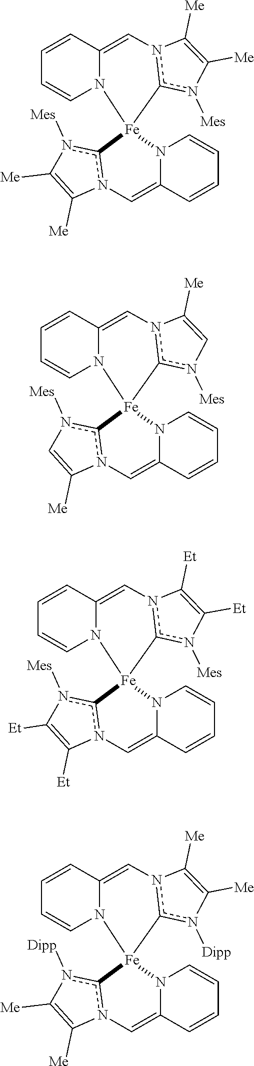

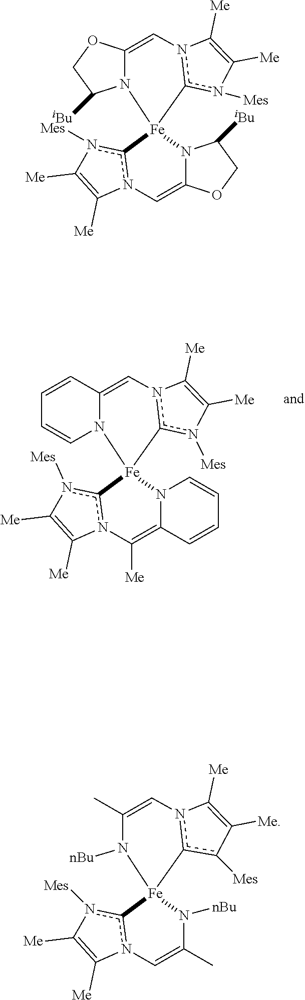

32. The iron-based complex of any one of claims 1 to 31, wherein the complex is wax-protected.

33. Use of the iron-based complex of any one of claims 1 to 31 for catalyzing a hydrosilylation reaction.

34. The use of claim 33, wherein the hydrosilylation reaction comprises the hydrosilylation of a ketone or an aldehyde.

35. The use of claim 34, wherein the hydrosilylation reaction comprises reacting the ketone or aldehyde with a hydrosilane selected from the group consisting of PhSiH₃, Me (OEt)₂SiH, (EtO)₃SiH, Ph₂SiH₂, Et₃SiH, polymethylhydrosiloxane (PMHS), 1,1,3,3-tetramethyldisiloxane (TMDS), and any mixture thereof.

36. The use of claim 34, wherein the hydrosilylation reaction comprises reacting the ketone or aldehyde with a hydrosilane selected from the group consisting of polymethylhydrosiloxane (PMHS), 1,1,3,3-tetramethyldisiloxane (TMDS), and any mixture thereof.

37. The use of claim 34, wherein the hydrosilylation reaction comprises reacting the ketone or aldehyde with polymethylhydrosiloxane (PMHS).

38. The use of any one of claims 34 to 37, wherein the hydrosilylation reaction is performed at ambient temperature.

39. The use of claim 34 or 38, wherein the hydrosilylation reaction comprises reacting a liquid ketone or aldehyde with a hydrosilane selected from the group consisting of polymethylhydrosiloxane (PMHS), 1,1,3,3-tetramethyldisiloxane (TMDS), and any mixture thereof, and the hydrosilylation reaction is performed in the absence of any solvent.

40. The use of any one of claims 34 to 39, wherein the ketone is of formula R(CO)R′ and the aldehyde is of formula RCHO, where:
R and R′ are independently selected from the group consisting of linear or branched C₁-C10alkyl, linear or branched C₂-C₁₀alkylenyl, C₃-C₁₀cycloalkyl, 4-to 6-membered heterocycloalkyl, C₆-C₁₀aryl, and 6 to 10-membered heteroaryl; or in the ketone R (CO) R′, R and R′ can form with the carbon atom of the CO group, a saturated or unsaturated C₄-C₁₀cycloalkyl or 5-to 10-membered heterocycloalkyl, and
the alkyl, cycloalkyl, heterocycloalkyl, aryl and heteroaryl are optionally substituted with one or more substituents selected from the group consisting of halogen, linear or branched C₁-C₆alkyl, linear or branched C₁-C₆alkoxy, NH₂, CN, and NO₂.

41. The use of claim 40, wherein R and R′ independently represent Me, Et, iPr, tBu, cyclopropyl, C₆H₅, C₆H₄F, C₆H₄Cl, C₆H₄Br, C₆H₄I, C₆H₄Me, C₆H₄OMe, C₆H₄NH₂, C₆H₄NO₂, C₆H₂Me₃, or R and R′ form together with the carbon to which they are attached a group C₆H₁₀.

42. The use of any one of claims 34 to 41, wherein a loading of the iron-based complex for the hydrosilylation reaction is less than 0.1 mol % based on the molar concentration of ketone or aldehyde.

43. The use of any one of claims 34 to 42, wherein a loading of the iron-based complex for the hydrosilylation is about 0.05 mol % based on the molar concentration of ketone or aldehyde.

44. A method for preparing a silyl ether from a ketone or an aldehyde comprising:
hydrosilylation of the ketone or aldehyde in the presence of the iron-based complex as defined in any one of claims 1 to 31 to form the silyl ether.

45. A method for synthesizing an alcohol from a ketone or an aldehyde comprising:
hydrosilylation of the ketone or aldehyde in the presence of the iron-based complex as defined in any one of claims 1 to 31 to form a silyl ether; and
hydrolyzing the silyl ether to obtain the alcohol.

46. The method of claim 45, wherein hydrolyzing is performed under basic conditions.

47. The method of any one of claims 44 to 46, wherein the hydrosilylation comprises reacting the ketone or aldehyde with a hydrosilane selected from the group consisting of PhSiH₃, Me(OEt)₂SiH, (EtO)₃SiH, Ph₂SiH₂, Et₃SiH, polymethylhydrosiloxane (PMHS), 1,1,3,3-tetramethyldisiloxane (TMDS), and any mixture thereof.

48. The method of any one of claims 44 to 46, wherein the hydrosilylation comprises reacting the ketone or aldehyde with a hydrosilane selected from the group consisting of polymethylhydrosiloxane (PMHS), 1,1,3,3-tetramethyldisiloxane (TMDS), and any mixture thereof.

49. The method of any one of claims 44 to 46, wherein the hydrosilylation comprises reacting the ketone or aldehyde with polymethylhydrosiloxane (PMHS).

50. The method of any one of claims 44 to 49, wherein the hydrosilylation is performed at ambient temperature.

51. The method of any one of claims 44 to 46 and 50, wherein the hydrosilylation comprises reacting a liquid ketone or aldehyde with a hydrosilane selected from the group consisting of polymethylhydrosiloxane (PMHS), 1,1,3,3-tetramethyldisiloxane (TMDS), and any mixture thereof, and the hydrosilylation reaction is performed in the absence of any solvent.

52. The method of any one of claims 44 to 49, wherein the ketone or aldehyde is solid at ambient temperature and the hydrosilylation is performed in the presence of a solvent.

53. The method of any one of claims 44 to 52, wherein the ketone is of formula R(CO)R′ and the aldehyde is of formula RCHO, where:
R and R′are independently selected from the group consisting of linear or branched C₁-C₁₀alkyl, linear or branched C₂-C₁₀alkylenyl, C₃-C₁₀cycloalkyl, 4-to 6-membered heterocycloalkyl, C₆-C₁₀aryl, and 6 to 10-membered heteroaryl; or in the ketone R(CO)R′, R and R′ can form with the carbon atom of the CO group, a saturated or unsaturated C₄-C₁₀cycloalkyl or 5-to 10-membered heterocycloalkyl, and
the alkyl, cycloalkyl, heterocycloalkyl, aryl and heteroaryl are optionally substituted with one or more substituents selected from the group consisting of halogen, linear or branched C₁-C₆alkyl, linear or branched C₁-C₆alkoxy, NH₂, CN, and NO₂.

54. The method of claim 53, wherein R and R′ independently represent Me, Et, iPr, tBu, cyclopropyl, C₆H₅, C₆H₄F, C₆H₄Cl, C₆H₄Br, C₆H₄I, C₆H₄Me, C₆H₄OMe, C₆H₄NH₂, C₆H₄NO₂, C₆H₂Me₃, or R and R′ form together with the carbon to which they are attached a group C₆H₁₀.

55. The method of any one of claims 44 to 54, wherein a loading of the iron-based complex for the hydrosilylation is less than 0.1 mol % based on the molar concentration of ketone or aldehyde.

56. The method of any one of claims 44 to 54, wherein a loading of the iron-based complex for the hydrosilylation is about 0.05 mol % based on the molar concentration of ketone or aldehyde.

57. A process for preparing the iron-based complex of any one of claims 1 to 31, comprising
reacting a compound of formula (II) 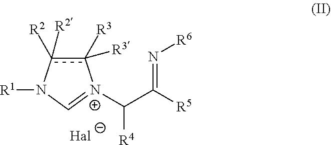
wherein [symbol]
represents a single bond or a double bond, and when [symbol] represents a double bond then R²′ and R³′ are absent;
R¹, R², R²′, R³, R³′, R⁵ , and R⁶ are as defined in any one of claims 1 to 26 and R⁴ is H;
with an iron salt.

58. The process of claim 57, wherein the reaction is performed in the presence of a base.

59. The process of claim 58, wherein the base is selected from the group consisting of BuLi, PhLi, Lithium diisopropylamide (LDA), KBn, NaH, KH, Lithium HexaMethylDiSilazid (LiHMDS), and Potassium HexaMethylDiSilazide (KHMDS).

60. The process of any one of claims 57 to 59, wherein the iron salt is selected from the group consisting of FeCl₂, FeBr₂, Fe(HMDS)₂, Fe(OAc)₂, Fe(OTf)₂, and FeCl₂(THF)_{1.5}.

61. The process of any one of claims 57 to 60, wherein the reaction is performed at ambient temperature.

62. The process of any one of claims 57 to 61, wherein the reaction is performed in the presence of a solvent selected from the group consisting of toluene, THF, Et₂O, dioxane, benzene, DME, xylene, mesitylene, and any mixture thereof.

63. The process of any one of claims 57 to 62, wherein the compound of formula (II) is such that R⁵ and R⁶ are as defined in any one of claims 24 to 26, and the compound of formula (II) is obtained by reacting a compound of formula (III) 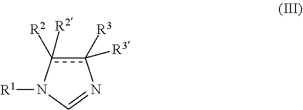
wherein [symbol]
represents a single bond or a double bond, and when [symbol] represents a double bond then R²′ and R³′ are absent;
and R¹, R², R²′, R³ and R³′ being as defined in any one of claims 1 to 20;
with a compound of formula (IV) 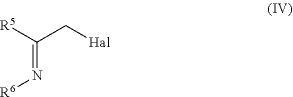
wherein R⁵ and R⁶ are as defined in any one of claims 24 to 26.

64. The process of claim 63, wherein the reaction between the compound of formula (III) and (IV) is performed under heating.

65. The process of claim 63 or 64, wherein the reaction between the compound of formula (III) and (IV) is performed in the presence of a solvent.

66. The process of claim 63 or 64, wherein the reaction between the compound of formula (III) and (IV) is performed in the presence of a solvent under reflux.

67. The process of claim 65 or 68, wherein the solvent selected from the group consisting of THF, other, toluene, benzene, DME, and any mixture thereof.

68. The process of any one of claims 63 to 67, wherein the compound of formula (IV) is a compound of formula (IVa) 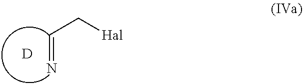
wherein 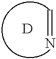
represents a moiety selected from the group consisting of 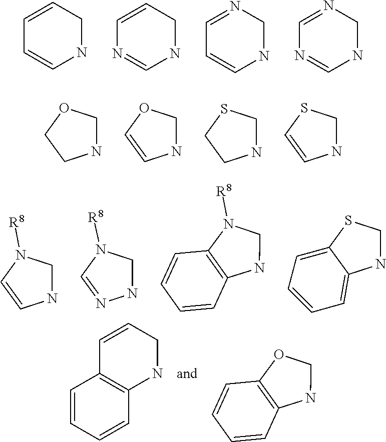
wherein R⁸ is a linear or branched C₁-C₆alkyl or C₆-C₁₄aryl and the moiety is optionally substituted with one or more substituents selected from the group consisting of halogen, linear or branched C₁-C₆alkyl, linear or branched C₂-C₆alkenyl, C₃-C₁₀cycloalkyl, C₃-C₁₀cycloalkenyl, linear or branched C₁-C₆alkoxy, C₃-C₁₀cycloalkoxy, C₆-C₁₄aryloxy, C₆-C₁₄aryl, NHR⁷, NR⁷R⁷′, C(O)OR⁷, C(O)NH₂, C(O)NHR⁷, C(O)NR⁷R⁷, tetrahydrofuran-2-yl, tetrahydrofuran-3-yl, NO₂, CN, NH₂, OCF₃, and CF₃, where R⁷and R⁷′ are as defined in claim 1.

69. The process of claim 67, wherein the moiety is substituted with a linear or branched C₁-C₆alkyl.

70. The process of claim 69, wherein the moiety is unsubstituted.

71. The process of any one of claims 57 to 70, further comprising reacting the iron-based complex where R⁴ is H with a compound of formula R⁹-LG where LG is a leaving group and R⁹ is a linear or branched C₁-C₁₈alkyl, or C₃-C₆cycloalkyl, and then with a base, to obtain the iron-based complex where R⁴ is linear or branched C₁-C₁₈alkyl, or C₃-C₆cycloalkyl.

72. The process of claim 71, wherein LG is selected from the group consisting of OTf, halogen, OTs, and any mixture thereof.

73. The process of claim 71 or 72, wherein the base is selected from the group consisting of BuLi, PhLi, Lithium diisopropylamide (LDA), KBn, NaH, KH, and alkali metal bis (trimethylsilyl) amide (HMDS).
